(19) United States
(12) Patent Application Publication
(10) Pub. No.: US 2023/0312857 A1
(43) Pub. Date: Oct. 5, 2023

(54) NANOPORE DEVICES INCLUDING BARRIERS USING POLYMERS WITH END GROUPS, AND METHODS OF MAKING THE SAME

(71) Applicant: Illumina Cambridge Limited, Cambridge (GB)

(72) Inventors: Oliver Uttley, Cambridge (GB); Davide Garoldini, Cambridge (GB); Istvan Kocsis, Cambridge (GB); Charlotte Vacogne, Cambridge (GB); Antonio Conde-Gonzalez, Cambridge (GB); Alexandre Richez, Cambridge (GB); Miguel Angel Aleman Garcia, Cambridge (GB); Yuliia Vyborna, Sawston (GB)

(73) Assignee: Illumina Cambridge Limited, Cambridge (GB)

(21) Appl. No.: 18/193,517

(22) Filed: Mar. 30, 2023

Related U.S. Application Data
(60) Provisional application No. 63/325,737, filed on Mar. 31, 2022.

(51) Int. Cl. C08J 9/228; C08J 9/40

(52) U.S. Cl. CPC C08J 9/228; C08J 9/40; C08J 2205/042; C08J 2353/00

(57) ABSTRACT

Nanopore devices including barriers using polymers with end groups, and methods of making the same, are provided herein. In some examples, a barrier between first and second fluids is provided. The barrier may be suspended by a barrier support defining an aperture. The barrier may include one or more layers suspended across the aperture and including molecules of a block copolymer. Each molecule of the block copolymer may include one or more hydrophilic blocks having an approximate length A and one or more hydrophilic blocks having an approximate length B. The hydrophilic blocks may form outer surfaces of the barrier and the hydrophobic blocks being located within the barrier. End groups may be coupled to ends of the hydrophilic blocks that form outer surfaces of the barrier. The end groups may have a different hydrophilicity than the hydrophilic blocks.

### CROSS-REFERENCE TO RELATED APPLICATIONS

This application claims the benefit of U.S. Provisional Patent Application No. 63/325,737, filed Mar. 31, 2022 and entitled “NANOPORE DEVICES INCLUDING BARRIERS USING POLYMERS WITH END GROUPS, AND METHODS OF MAKING THE SAME”, the entire contents of which are incorporated by reference herein.

### FIELD

This application relates to devices that include barriers.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein pore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Nanopore devices including barriers using polymers with end groups, and methods of making the same, are provided herein.

In some examples, a barrier between first and second fluids is provided. The barrier may be suspended by a barrier support defining an aperture. The barrier may include one or more layers suspended across the aperture and including molecules of a block copolymer. Each molecule of the block copolymer may include one or more hydrophilic blocks having an approximate length A and one or more hydrophilic blocks having an approximate length B. The hydrophilic blocks may form outer surfaces of the barrier and the hydrophobic blocks being located within the barrier. End groups may be coupled to ends of the hydrophilic blocks that form outer surfaces of the barrier. The end groups may have a different hydrophilicity than the hydrophilic blocks.

In some examples, the end groups are selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

In some examples, the hydrophobic blocks comprise a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

In some examples, the block copolymer is a diblock copolymer. In some examples, the hydrophobic block is polybutadiene (PBd). In some examples, the barrier has a thickness of approximately 2A+2B.

In some examples, the block copolymer is a triblock copolymer having two hydrophilic blocks and one hydrophobic block. In some examples, the hydrophobic block is poly(isobutylene) (PIB). In some examples, the barrier has a thickness of approximately 2A+B.

In some examples, the block copolymer is a triblock copolymer having two hydrophobic blocks and one hydrophilic block. In some examples, the barrier has a thickness of approximately A+2B.

In some examples, the barrier further includes a nanopore disposed therein and providing contact between the first fluid and the second fluid.

Some examples herein provide a barrier that includes a at least one layer that includes a plurality of molecules. Each of the molecules may include first and second hydrophilic blocks, first and second end groups, and a hydrophobic block. The hydrophobic block may be disposed between and coupled to the first and second hydrophilic blocks. The first and second end groups respectively may be coupled to ends of the first and second hydrophilic blocks and may have a different hydrophilicity than the first and second hydrophilic blocks. The first and second end groups may form first and second outer surfaces of the barrier. The hydrophobic blocks may be within the barrier.

In some examples, the first and second hydrophilic blocks each include poly(ethylene oxide) (PEO). In some examples, the first and second hydrophilic blocks each include between about 2 and about 100 PEO repeating units. In some examples, the first and second hydrophilic blocks each include between about 2 and about 12 PEO repeating units. In some examples, the first and second hydrophilic blocks each include between about 2 and about 4 PEO repeating units. In some examples, the first and second hydrophilic blocks each include between about 3 and about 9 PEO repeating units. In some examples, the first and second hydrophilic blocks each include between about 9 and about 12 PEO repeating units.

In some examples, the hydrophobic block includes poly(dimethylsiloxane) (PDMS) or poly(isobutylene) (PIB). In some examples, the hydrophobic block includes about 2 to about 100 PDMS repeating units. In some examples, the hydrophobic block includes about 13 to about 44 PDMS repeating units, or about 30 to about 44 PDMS repeating units. In some examples, the hydrophobic block includes about 2 to about 100 PIB repeating units. In some examples, the hydrophobic block includes about 13 to about 44 PIB repeating units, or about 30 to about 44 PIB repeating units.

In some examples, the hydrophobic block is coupled to the first and second hydrophilic blocks via respective sulfide, ether, ester, alkyl, or triazole bonds.

In some examples, the first and second end groups independently are selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

In some examples, the barrier further includes a nanopore. In some examples, the nanopore includes α-hemolysin or MspA.

Some examples herein provide a barrier that includes at least one layer including a plurality of molecules. Each of the molecules may include first and second ionic end groups and a hydrophobic block. The hydrophobic block may be disposed between and coupled to the first and second ionic end groups. The ionic end groups may form first and second outer surfaces of the barrier. The hydrophobic blocks may be within the barrier.

In some examples, the first ionic end group and the second ionic end group each include a zwitterion. In some examples, the first ionic end group and the second ionic end group are selected from the group consisting of: 2-methacryloyloxyethyl phosphorylcholine, 3-[dimethyl-[2-(2-methylprop-2-enoyloxy)ethyl]azaniumyl]propane-1-sulfonate (DMAPS), 3-{[3-(acryloylamino)propyl](dimethyl)ammonio}propanoate, and 3-{[3-(acryloylamino)propyl](dimethyl)ammonio}-1-propanesulfonate.

In some examples, the first ionic end group and the second ionic end group each include a cation. In some examples, the first ionic end group and the second ionic end group each include 2-(trimethylammonio)ethyl methacrylate.

In some examples, the first ionic end group and the second ionic end group each include an anion. In some examples, the first ionic end group and the second ionic end group each include 3-sulfopropyl acrylate, 2-propene-1-sulfonate, or vinylphosphonic acid.

In some examples, the hydrophobic block includes poly(dimethylsiloxane) (PDMS) or poly(isobutylene) (PIB). In some examples, the PDMS includes about 2 to 100 PDMS repeating units. In some examples, the PDMS includes about 13 to about 44 PDMS repeating units, or about 30 to about 44 PDMS repeating units. In some examples, the PIB includes about 2 to about 100 PIB repeating units. In some examples, the PIB includes about 13 to about 44 PIB repeating units, or about 30 to about 44 PIB repeating units.

In some examples, the first and second ionic end groups are each coupled to the hydrophobic block via the product of a hydrosilylation, amine-ester coupling, CuAAC click chemistry, DBCO-azide, a thiol-Michael addition, or a thiol-ene click reaction.

In some examples, the barrier further includes a nanopore. In some examples, the nanopore includes α-hemolysin or MspA.

Some examples herein provide a barrier that includes a first layer including a first plurality of molecules, and a second layer including a second plurality of the molecules. Each of the molecules may include a hydrophilic block, a hydrophobic block, and an end group. The hydrophilic block may be coupled to the hydrophobic block. The end group may be coupled to an end of the hydrophilic block and may have a different hydrophilicity than the hydrophilic block. The end groups may form first and second outer surfaces of the barrier. The hydrophobic blocks of the first and second pluralities of the molecules may contact one another within the barrier.

In some examples, the hydrophilic block includes poly(ethylene oxide) (PEO). In some examples, the hydrophilic block includes between about 2 and about 100 PEO repeating units. In some examples, the hydrophilic block includes between about 2 and about 12 PEO repeating units, for example between about 2 and about 9 PEO repeating units.

In some examples, the hydrophobic block includes poly(dimethylsiloxane) (PDMS) or poly(isobutylene) (PIB). In some examples, the hydrophobic block includes between about 2 and about 100 PDMS repeating units. In some examples, the hydrophobic block includes between about 14 and about 44 PDMS repeating units. In some examples, the hydrophobic block includes between about 14 and about 26 PDMS repeating units, or between about 26 and about 44 PDMS repeating units. In some examples, the hydrophobic block includes between about 2 and about 100 PIB repeating units. In some examples, the hydrophobic block includes between about 14 and about 44 PIB repeating units. In some examples, the hydrophobic block includes between about 14 and about 26 PIB repeating units, or between about 26 and about 44 PIB repeating units.

In some examples, the end group is selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

In some examples, the barrier further includes a nanopore. In some examples, the nanopore includes α-hemolysin or MspA.

Some examples herein provide a barrier that includes a first layer including a first plurality of molecules, and a second layer including a second plurality of the molecules. Each of the molecules may include first and second hydrophobic blocks, a hydrophilic block, and first and second end groups. The hydrophilic block may be disposed between and coupled to the first and second hydrophobic blocks. The first and second end groups respectively may be coupled to ends of the first and second hydrophobic blocks and may have a different hydrophobicity than the first and second hydrophobic blocks. The hydrophilic blocks of the first plurality of the molecules may form a first outer surface of the barrier. The hydrophilic blocks of the second plurality of the molecules may form a second outer surface of the barrier. The first and second end groups of the first and second pluralities of the molecules may contact one another within the barrier.

In some examples, the hydrophilic block includes poly(ethylene oxide) (PEO). In some examples, the hydrophilic block includes between about 2 and about 100 PEO repeating units. In some examples, the hydrophilic block includes between about 2 and about 13 PEO repeating units, e.g., between about 7 and about 13 PEO repeating units, or between about 2 and about 7 PEO repeating units.

In some examples, the first and second hydrophobic blocks each include poly(dimethylsiloxane) (PDMS) or poly(isobutylene) (PIB). In some examples, the first and second hydrophobic blocks each include between about 2 and about 100 PDMS repeating units. In some examples, the first and second hydrophobic blocks each include between about 14 and about 44 PDMS repeating units, e.g., between about 14 and about 26 PDMS repeating units, or between about 26 PDMS repeating units and about 44 PDMS repeating units. In some examples, the first and second hydrophobic blocks each include between about 2 and about 100 PIB repeating units. In some examples, the first and second hydrophobic blocks each include between about 14 and about 44 PIB repeating units, e.g., between about 14 and about 26 PIB repeating units, or between about 26 PIB repeating units and about 44 PIB repeating units.

In some examples, the first and second end groups include a lower alkyl (C₁₋₄ alkyl) or an aryl group, a polycyclic aromatic hydrocarbon, a fluorinated alkyl, or a fluorinated aryl group. In some examples, the lower alkyl includes a methyl, ethyl, propyl, or n-butyl group.

In some examples, the barrier further includes a nanopore. In some examples, the nanopore includes α-hemolysin or MspA.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example nanopore composition and device including a barrier using a polymer with end groups.

FIGS. 2A-2B schematically illustrate plan and cross-sectional views of further details of the nanopore composition and device of FIG. 1.

FIG. 3 schematically illustrates a cross-sectional view of a barrier including an example diblock copolymer with end groups.

FIG. 4 schematically illustrates a cross-sectional view of a barrier including an example triblock copolymer with end groups.

FIG. 5 schematically illustrates a cross-sectional view of a barrier including an example polymer with ionic end groups.

FIG. 6 schematically illustrates a cross-sectional view of a barrier including another example triblock copolymer with end groups.

FIGS. 7A-7C schematically illustrate further details of barriers using polymers which may be included in the nanopore composition and device of FIG. 1 and used in barriers such as described with reference to FIGS. 3-6.

FIGS. 8A-8C schematically illustrate example schemes for preparing triblock copolymers for use in the nanopore composition and device of FIG. 1 and barriers such as described with reference to FIGS. 3-6.

FIG. 9 schematically illustrates a cross-sectional view of an example use of the composition and device of FIG. 1.

FIG. 10 schematically illustrates a cross-sectional view of another example use of the composition and device of FIG. 1.

FIG. 11 schematically illustrates a cross-sectional view of another example use of the composition and device of FIG. 1.

FIG. 12 schematically illustrates a cross-sectional view of another example use of the composition and device of FIG. 1.

FIG. 13 illustrates the voltage breakdown waveform used to assess polymeric membrane stability.

FIG. 14A illustrates a plot of the measured breakdown voltage of example barriers.

FIG. 14B illustrates a plot of the respective currents through the barriers of FIG. 14A with a nanopore inserted therein.

FIG. 14C illustrates a plot of the respective noise in current through the barriers of FIG. 14A with a nanopore inserted therein.

FIG. 15A illustrates a plot of the measured breakdown voltage of additional example barriers.

FIG. 15B illustrates a plot of the respective currents through the barriers of FIG. 15A with a nanopore inserted therein.

FIG. 15C illustrates a plot of the respective noise in current through the barriers of FIG. 15A with a nanopore inserted therein.

FIG. 16 illustrates a plot of barrier noise and half-life voltage as a function of the number of repeat units in the hydrophobic block.

FIG. 17 schematically illustrates a cross-sectional view of another example use of the composition and device of FIG. 1.

FIG. 18 illustrates a flow of operations for forming a device such as illustrated in FIG. 1.

FIG. 19 illustrates a plot describing the breakdown voltage measured for membranes formed in accordance with examples herein.

FIG. 20 illustrates a plot of MspA nanopore/membrane construct stability under certain conditions.

### DETAILED DESCRIPTION

Nanopore devices including barriers using polymers with end groups, and methods of making the same, are provided herein.

For example, nanopore sequencing may utilize a nanopore that is inserted into a barrier, and that includes an aperture through which ions and/or other molecules may flow from one side of the barrier to the other. Circuitry may be used to detect a sequence, for example a sequence of nucleotides, e.g., during sequencing-by-synthesis (SBS) in which, on a first side of the barrier, a polymerase adds the nucleotides to a growing polynucleotide in an order that is based on the sequence of a template polynucleotide to which the growing polynucleotide is hybridized. The sensitivity of the circuitry may be improved by using fluids with different compositions on respective sides of the barrier, for example to provide suitable electron transport for detection on one side of the barrier, while suitably promoting activity of the polymerase on the other side of the barrier. The difference in fluidic compositions may generate an osmotic pressure that may weaken the barrier, and thus increase the likelihood that the barrier may break or leak during normal use. However, it may be difficult to insert nanopores into barriers that are too strong.

As provided herein, barriers for use in nanopore devices may include polymers that provide suitable stability characteristics for long-term use of the device, and that also facilitate nanopore insertion so as to increase the number of usable devices during production. As explained in greater detail below, in some examples, the present polymers may include a hydrophilic block coupled to a hydrophobic block (e.g., may include a diblock copolymer). In other examples, the present polymers may include a hydrophilic block coupled between two hydrophobic blocks, or a hydrophobic block coupled between two hydrophilic blocks (e.g., may include a triblock copolymer). In still other examples, the present polymers may include a hydrophobic block and may not include any hydrophilic block(s). As provided herein, throughout the various examples the respective ends of the hydrophobic block(s), the hydrophilic block(s), or both the hydrophilic block(s) and hydrophilic block(s) may include end groups which have different hydrophilicities than the blocks to which they are coupled. The end groups, as well as the respective lengths of the hydrophobic and/or hydrophilic blocks in the polymer, may be selected such that the polymers assemble into a barrier having suitable stability and usability, e.g., in nanopore sequencing.

First, some terms used herein will be briefly explained. Then, some example barriers using polymers with end groups, methods of making the same, and devices and methods using the same, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP.

Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NSSB protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105: 20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions or water-soluble molecules such as nucleotides and amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state membranes or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a membrane material that is not of biological origin (e.g., polymeric materials, synthetic phospholipids, solid-state membranes, or combinations thereof).

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

As used herein, a “polymeric membrane” or a “polymer membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “polymer membrane,” “membrane,” and “barrier” may be used interchangeably herein when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. The first portion optionally may include an end group with a hydrophilicity that is different than that of the first type of monomer, or the second portion optionally may include an end group with a hydrophilicity that is different than that of the second type of monomer, or the first portion optionally may include an end group with a hydrophilicity that is different than that of the first type of monomer and the second portion optionally may include an end group with a hydrophilicity that is different than that of the second type of monomer. The end groups of any hydrophilic blocks may be located at an outer surface of a barrier formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer (repeating unit) as one another, and the second block may include a different type of monomer (repeating unit). In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric membrane may depend, among other things, on the respective block lengths, the type(s) of monomers used in the different blocks, the relative hydrophilicities and hydrophobicities of the blocks, the composition of the fluid(s) within which the membrane is formed, and/or the density of the polymeric chains within the membrane. During formation of the membrane, these and other factors generate forces between molecules of the polymeric chains which laterally position and reorient the molecules in such a manner as to substantially minimize the free energy of the membrane. The membrane may be considered to be substantially “stable” once the polymeric chains have completed these rearrangements, even though the molecules may retain some fluidity of movement within the membrane.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic. Additionally, molecules including a hydrophobic polymer coupled to ionic end groups may be considered to be amphiphilic.

As used herein, a “solution” is intended to refer to a homogeneous mixture including two or more substances. In such a mixture, a solute is a substance which is uniformly dissolved in another substance referred to as a solvent. A solution may include a single solute, or may include a plurality of solutes. Additionally, or alternatively, a solution may include a single solvent, or may include a plurality of solvents. An “aqueous solution” refers to a solution in which the solvent is, or includes, water.

As used herein, the term “electroporation” means the application of a voltage across a membrane such that a nanopore is inserted into the membrane.

As used herein, “Cₐ to C_{b}” or “C_{α-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” or “C₁₋₄alkyl” group refers to all alkyl groups having from 1 to 4 carbons, that is, CH₃⁻, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may include 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” or “C₁₋₄alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, the term “linker” is intended to mean a molecule or molecules via which one element is attached to another element. For example, a linker may attach a first reactive moiety to a second reactive moiety. Linkers may be covalent, or may be non-covalent. Nonlimiting examples of covalent linkers include alkyl chains, sulfides, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the term “end group” is intended to mean a moiety that is located at the terminal end of an elongated molecule, such as a polymer. For example, an end group may be coupled to the terminal end of a polymer, and as such the end group may form the terminal end of the molecule that includes the polymer and the end group.

As used herein, the term “ionic group” is intended to mean a moiety that includes at least one charged atom. An ionic group may include a cation (positively charged atom), an anion (negatively charged atom), or both a cation and an anion in which case the ionic group may be referred to as “zwitterionic.” An ionic group may be associated with, but not covalently bound to, a counterion that balances the charge of the ionic group. For example, an ionic group including a cation may be associated with a negatively charged counterion that is not covalently bound to the ionic group. Or, for example, an ionic group including an anion may be associated with a positively charged counterion that is not covalently bound to the ionic group. The positively and negatively charged atoms of zwitterionic group may charge balance one another, so that the zwitterionic group overall is electrically balanced, that is, charge neutral.

As used herein, the term “linker” is intended to mean a moiety, molecule, or molecules via which one element is attached to another element. Linkers may be covalent, or may be non-covalent. Nonlimiting examples of covalent linkers include moieties such as alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the terms “PEO”, “PEG”, “poly(ethylene oxide)”, and “poly(ethylene glycol)” are intended to be used interchangeably and refer to a polymer that comprises —[CH₂—CH₂—O]ₙ—. In some examples, n is between about 2 and about 100.

As used herein, the term “barrier support” is intended to refer to a structure that can suspend a barrier. When the barrier includes a polymer membrane, the barrier support may be referred to as a “membrane support.” A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more First features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8; polytetrafluoroethylene (PTFE), poly(methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

Nanopore Devices Including Barriers Using Polymers with End Groups, and Methods of Making the Same

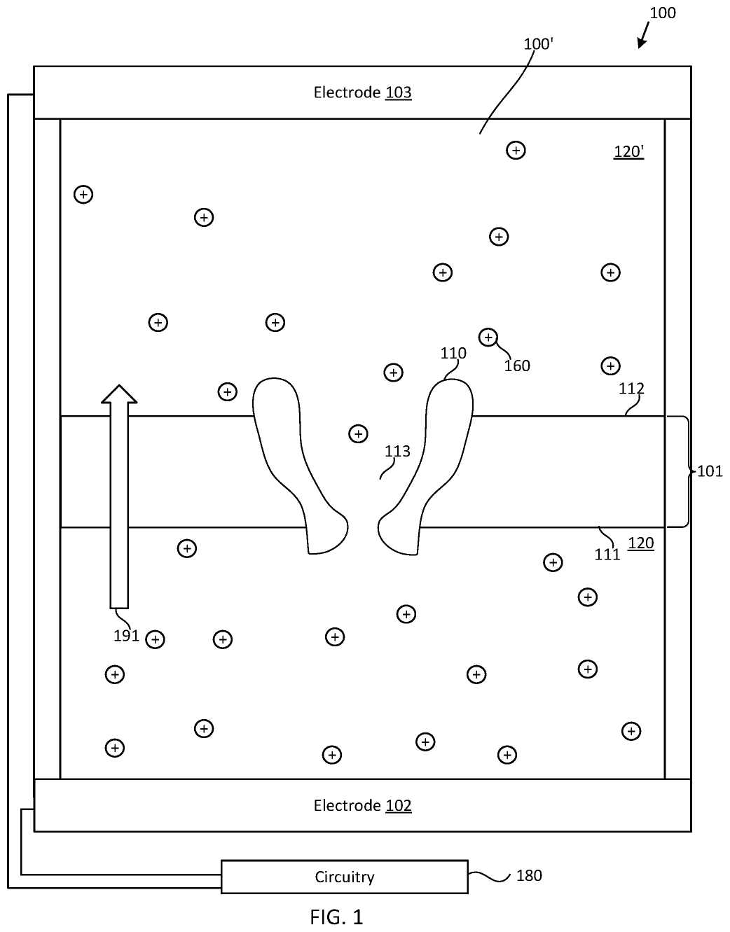

Some example devices including barriers using polymers with end groups, and methods of making the same, will be described with FIGS. 1, 2A-2B, 3, 4, 5, 6, 7A-7C, and 8A-8C.

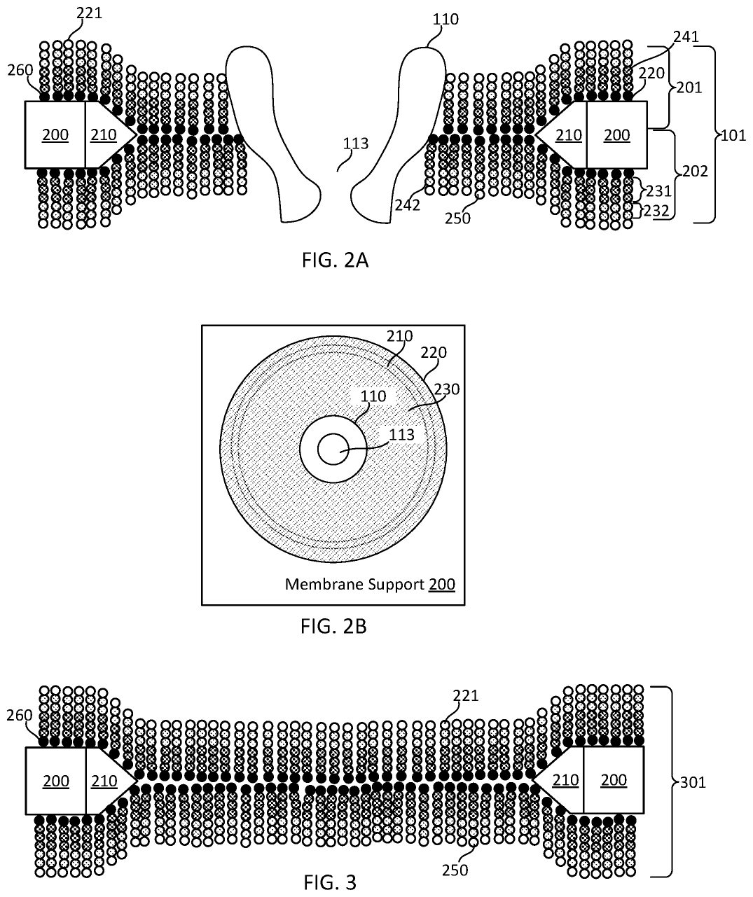

FIG. 1 schematically illustrates a cross-sectional view of an example nanopore composition and device 100 including a barrier using a polymer with end groups. Device 100 includes fluidic well 100′ including polymeric membrane (barrier) 101 having first (trans) side 111 and second (cis) side 112, first fluid 120 within fluidic well 100′ and in contact with first side 111 of the membrane, and second fluid 120′ within the fluidic well and in contact with the second side 112 of the membrane. Polymeric membrane 101 may have any suitable structure that normally inhibits passage of molecules from one side of the membrane to the other side of the membrane, e.g., that normally inhibits contact between fluid 120 and fluid 120′. Illustratively, polymeric membrane 101 may include a diblock or triblock copolymer including one or more end groups, or may include a hydrophobic polymer coupled to ionic end groups, and may have a structure such as described in greater detail below with reference to FIG. 2A-2B, 3, 4, 5, 6, or 7A-7C. As provided herein, the end group(s) of polymers in the polymeric membrane may be selected so as to provide membranes having enhanced stability and usability, and in some examples reduced length of the hydrophobic and/or hydrophilic block(s), as compared to membranes that do not include such end groups.

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that may be the same as, or different, than the first concentration. Any suitable salt or salts 160 may be used in first and second fluids 120, 120′, e.g., ranging from common salts to ionic crystals, metal complexes, ionic liquids, or even water-soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one nonlimiting example, the salt includes potassium chloride (KCl). It will also be appreciated that the first and second fluids optionally may include any suitable Combination of other solutes. Illustratively, first and second fluids 120, 120′ may include an aqueous buffer (such as N-(2-hydroxyethyl)piperazine-N′-2-ethanesulfonic acid (HEPES), commercially available from Fisher BioReagents).

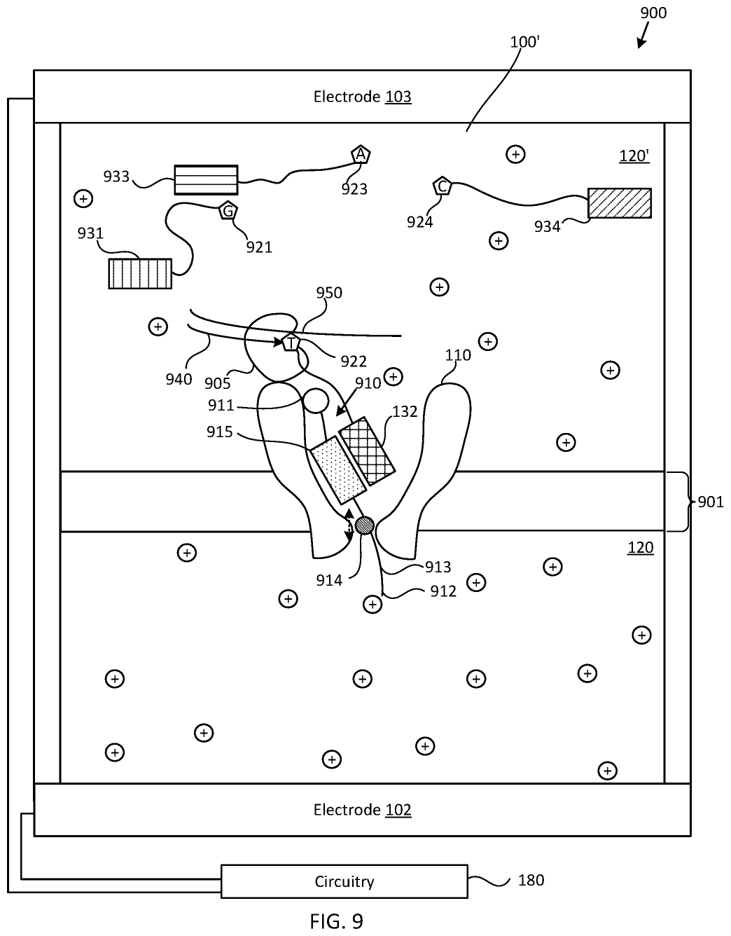

Still referring to FIG. 1, in some examples provided herein, device 100 optionally further may include nanopore disposed within barrier 101 and providing aperture 113 fluidically coupling first side 111 to second side 112. As such, aperture 113 of nanopore 110 may provide a pathway for fluid 120 and/or fluid 120′ to flow through barrier 101. For example, a portion of salt 160 may move from second side 112 of barrier 101 to first side 111 of the barrier through aperture 113. Nanopore 110 may include a solid-state nanopore, a biological nanopore (e.g., MspA such as illustrated in FIG. 1), or a biological and solid-state hybrid nanopore. Nonlimiting examples and properties of barriers and nanopores are described elsewhere herein, as well as in U.S. Pat. No. 9,708,655, the entire contents of which are incorporated by reference herein. In a manner such as illustrated in FIG. 1, device 100 optionally may include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used in any suitable application or context, including any suitable method or device for sequencing, e.g., polynucleotide sequencing. As provided herein, the polymer and end groups used in barrier 101 may be selected so as to provide the barrier with sufficient stability for use over a desired period of time, e.g., for use over the course of sequencing, e.g., sequencing a polynucleotide in a manner such as described with reference to FIGS. 9-12 and 17.

In some examples, polymeric membrane 101 between first and second fluids 120, 120′ includes a block copolymer with end groups. For example, FIGS. 2A-2B schematically illustrate plan and cross-sectional views of further details of the nanopore composition and device of FIG. 1. As illustrated in FIG. 2A, membrane 101 may include first layer 201 including a first plurality of block copolymer molecules 221 and second layer 202 including a second plurality of the block copolymer molecules. In the nonlimiting example illustrated in FIG. 2A, the copolymer is a diblock copolymer (AB), such that each molecule 221 includes a hydrophobic “B” block 231 (within which circles 241 with darker fill represent hydrophobic monomers) and a hydrophilic “A” block 232 (within which circles 242 with lighter fill represent hydrophilic monomers) coupled directly or indirectly thereto. One end of the hydrophilic A block 232 optionally may be coupled to end group 250 (represented by circle with white fill) and the other end of that A block may be coupled to hydrophobic block 231. End group 250 may have a different hydrophilicity than hydrophilic block 232. For example, end group 250 may include a different type of molecule than the hydrophilic monomers 242, that is, a molecule which is different than would occur at the end of hydrophilic A block 232 in the absence of the end group.

Additionally, or alternatively, one end of the hydrophobic block 231 optionally may be coupled to end group 260 (represented by circle with black fill) and the other end of that B block may be coupled to hydrophilic block 232. End group 260 may have a different hydrophilicity than hydrophobic block 231. For example, end group 260 may include a different type of molecule than the hydrophobic monomers 241, that is, a molecule which is different than would occur at the end of hydrophilic A block 232 in the absence of the end group. In other examples such as will be described with reference to FIGS. 4 and 6, the polymer instead may include a triblock copolymer (e.g., ABA or BAB, respectively) the terminal ends of which respectively may be coupled to end groups. In still other examples such as will be described with reference to FIG. 5, the polymer instead may include a hydrophobic polymer the ends of which are coupled to respective ionic groups.

In the example illustrated in FIG. 2A, end groups 250 coupled to the hydrophilic blocks 232 of the first plurality of molecules 221 may form a first outer surface of membrane 101, e.g., the surface of membrane 101 contacting fluid 120 on first side 111. End groups 250 coupled to the hydrophilic blocks 232 of the second plurality of molecules 221 may form a second outer surface of membrane 101, e.g., the surface of membrane 101 contacting fluid 120′ on second side 112. Additionally, or alternatively, end groups 260 coupled to the hydrophobic blocks 231 of the first and second pluralities of molecules 221 may contact one another within the membrane, otherwise the terminal ends of hydrophobic blocks 231 of the first and second pluralities of molecules 221 may contact one another within the membrane.

In the example illustrated in FIGS. 2A-2B, membrane 101 may be suspended using a barrier support, e.g., membrane support 200, defining aperture 230. For example, membrane support 200 may include a substrate having an aperture 230 defined therethrough, e.g., a substantially circular aperture, or an aperture having another shape. Additionally, or alternatively, the membrane support may include one or more features of a well in which the nanopore device is formed, such as a lip or ledge on either side of the well. Nonlimiting examples of materials which may be included in a barrier support are provided further above. An annulus 210 including hydrophobic (non-polar) solvent, and which also may include polymer chains and/or other compound(s), may adhere to membrane support 200 and may support a portion of membrane 101, e.g., may be located within barrier 101 (here, between layer 201 and layer 202). Additionally, annulus 210 may taper inwards in a manner such as illustrated in FIG. 2A. An outer portion of the molecules 221 of membrane 101 may be disposed on support 200 (e.g., the portion extending between aperture 230 and membrane periphery 220), while an inner portion of the molecules may form a freestanding portion of membrane 101 (e.g., the portion within aperture 210, a part of which is supported by annulus 210). Barrier 101 may be prepared, and nanopore 110 may be inserted into the freestanding portion of barrier 101, using operations such as described elsewhere herein. Although FIGS. 2A-2B illustrate nanopore 110 within barrier 101, it should be understood that the nanopore may be omitted, and that barrier 101 may be used for any suitable purpose. More generally, it should be appreciated that while the barriers described herein are particularly suitable for use with nanopores (e.g., for nanopore sequencing such as described with reference to FIGS. 9-12 and 17), the present barriers need not necessarily have nanopores inserted therein.

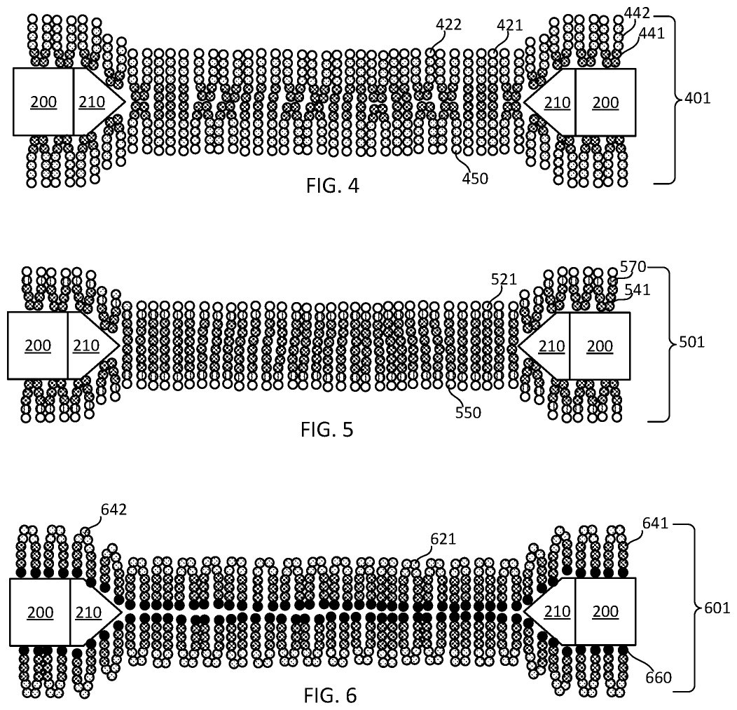

FIG. 3 schematically illustrates a cross-sectional view of a suspended barrier including an example diblock copolymer with end groups. As illustrated in FIG. 3, diblock copolymer membrane 301 may be configured similarly as membrane 101 described as reference to FIGS. 2A-2B, e.g., may include a diblock copolymer including layers within which the molecules of the diblock copolymer are oriented such that the hydrophobic “B” sections of the AB diblock copolymer are oriented towards each other and disposed within the membrane, while the hydrophilic “A” sections form the outer surfaces of the membrane. Suitable methods of forming suspended membranes on a membrane support are known in the art, such as “painting”, e.g., brush painting (manual), mechanical painting (e.g., using stirring bar), and bubble painting (e.g., using flow through the device). Nanopore 110 may be inserted into membrane 301 after the membrane is formed. Nonlimiting examples of techniques for inserting nanopore 110 into membrane 301 include electroporation, pipette pump cycle, and detergent assisted nanopore insertion. Tools for forming membranes using synthetic polymers and inserting nanopores in the membranes are commercially available, such as the Orbit 16 TC platform available from Nanion Technologies Inc. (California, USA).

Although FIGS. 2A and 3 illustrate devices and barriers that include a diblock copolymer, it will be appreciated that such devices and barriers may include other types of polymers, and that nanopores optionally may be inserted into such barriers. For example, FIG. 4 schematically illustrates a cross-sectional view of a barrier including an example triblock copolymer with end groups. FIG. 4 illustrates membrane 401 including molecules 421, 422 of an ABA triblock copolymer, which may be suspended using barrier support 200 and annulus in a manner such as described with reference to FIGS. 2A-2B. Each of the molecules 421, 422 includes first and second hydrophilic A blocks 442, first and second end groups 450, and hydrophobic B blocks 441 coupled to and between the first and second hydrophilic A blocks 442. The first and second end groups 450 respectively are coupled to ends of the first and second hydrophilic blocks 442. The first and second end groups 450 may have a different hydrophilicity than the first and second hydrophilic blocks 442. For example, end groups 450 may include a different type of molecule than the repeating units of hydrophilic monomer forming hydrophilic blocks 442, that is, a molecule which is different than would occur at the ends of hydrophilic A block 442 in the absence of the end groups 450. In the example shown in FIG. 4, each individual ABA molecule may be in one of two arrangements. For example, ABA molecules 421 may extend through the layer in a linear fashion, with an “A” block on each side of the membrane and the “B” block in the middle of the membrane. Or, for example, ABA molecules 422 may extend to the middle of the membrane and then fold back on themselves, so that both “A” blocks are on the same side of the membrane and the “B” block is in the middle of the membrane. In regions where ABA molecules are inhibited from fully extending, e.g., at annulus 210 or barrier 200, the ABA molecules may take on folded arrangement 422. For either arrangement, end groups 250 form first and second outer surfaces of barrier 401, and the hydrophobic blocks 441 contact one another within the barrier. Accordingly, in this example, barrier 401 may be considered to be partially a single layer and partially a bilayer. In other examples (not specifically illustrated) in which barrier 401 substantially includes molecules 421 which extend through the barrier in linear fashion, barrier 401 may substantially be a monolayer. In still other examples (not specifically illustrated) in which barrier 401 substantially includes molecules 422 which extend to approximately the middle of the barrier and then fold back on themselves, barrier 401 may substantially be a bilayer. A nanopore, not specifically, shown, optionally may be inserted into any of such options for barrier 1501 in a manner similar to that described elsewhere herein, e.g., as illustrated in FIGS. 2A-2B.

In another example, FIG. 5 schematically illustrates a cross-sectional view of a barrier including an example polymer with ionic end groups. FIG. 5 illustrates membrane 501 including molecules 521 including first and second ionic end groups 550 and hydrophobic block 541, which may be suspended using barrier support 200 and annulus in a manner such as described with reference to FIGS. 2A-2B. The hydrophobic block 541 may be disposed between and coupled to the first and second ionic end groups. Such coupling may be direct or indirect. In the nonlimiting example illustrated in FIG. 5, end groups 550 are coupled to respective ends of hydrophobic block 541 via linkers 570. The first and second end groups 550 may have a different hydrophilicity than hydrophobic block 541. For example, end groups 550 may include a different type of molecule than the repeating units of hydrophobic monomer forming hydrophobic blocks 541, that is, a molecule which is different than would occur at the ends of hydrophobic block 541 in the absence of the end groups 550.

In the example shown in FIG. 5, each individual molecule may be in one of two arrangements. For example, in the suspended portion of membrane 501 (within aperture 230), molecules 521 may extend through the layer in a linear fashion, with an ionic end group 550 on each side of the membrane and the hydrophobic “B” block in the middle of the membrane. Or, for example, in regions where molecules 521 are inhibited from fully extending, e.g., at annulus 210 or barrier 200, the molecules may take on a folded arrangement similar to that described with reference to FIG. 4. In such example, barrier 501 may be considered to be substantially a monolayer, because the suspended portion is substantially a monolayer. For either arrangement, ionic end groups 550 form first and second outer surfaces of barrier 501, and the hydrophobic blocks 541 contact one another within the barrier. In other examples (not specifically illustrated), the suspended portion of barrier 501 may include a mixture of molecules 521 which extend through the barrier in linear fashion and molecules which extend to approximately the middle of the barrier and then fold back on themselves in a manner such as molecules 422 described with reference to FIG. 4, in which case barrier 501 may be considered to be partially a single layer and partially a bilayer. In still other examples, barrier 501 substantially may include molecules which extend to approximately the middle of the barrier and then fold back on themselves in a manner such as molecules 422 described with reference to FIG. 4, in which case barrier 501 may be considered to be substantially a bilayer. A nanopore, not specifically, shown, optionally may be inserted into any of such options for barrier 501 in a manner similar to that described elsewhere herein, e.g., as illustrated in FIGS. 2A-2B.

FIG. 6 schematically illustrates a cross-sectional view of a barrier including another example triblock copolymer with end groups. FIG. 6 illustrates membrane 601 including molecules 621 of a BAB triblock copolymer including hydrophilic “A” block 642, first and second hydrophobic “B” blocks 641, and first and second end groups 660, which may be suspended using barrier support 200 and annulus in a manner such as described with reference to FIGS. 2A-2B. The hydrophilic block 642 is disposed between and coupled to the first and second hydrophobic blocks 641. First and second end groups 660 respectively are coupled to ends of the first and second hydrophobic blocks 641. The first and second end groups 660 may have a different hydrophilicity than hydrophobic blocks 641. For example, end groups 660 may include a different type of molecule than the repeating units of hydrophobic monomer forming hydrophobic blocks 641, that is, a molecule which is different than would occur at the ends of hydrophobic block 641 in the absence of the end groups 660. In this example, membrane 601 may have a bilayer architecture with the “B” blocks 641 oriented towards each other. The end groups 660 coupled to the hydrophobic blocks of the BAB molecules generally may be located approximately in the middle of membrane 601, the molecules then extend towards either outer surface of the membranes, and then fold back on themselves. As such, both “B” blocks are located in the middle of the membrane and the “A” blocks form the first and second outer surfaces of the membrane. A nanopore, not specifically, shown, optionally may be inserted into any of such options for barrier 601 in a manner similar to that described elsewhere herein, e.g., as illustrated in FIGS. 2A-2B.

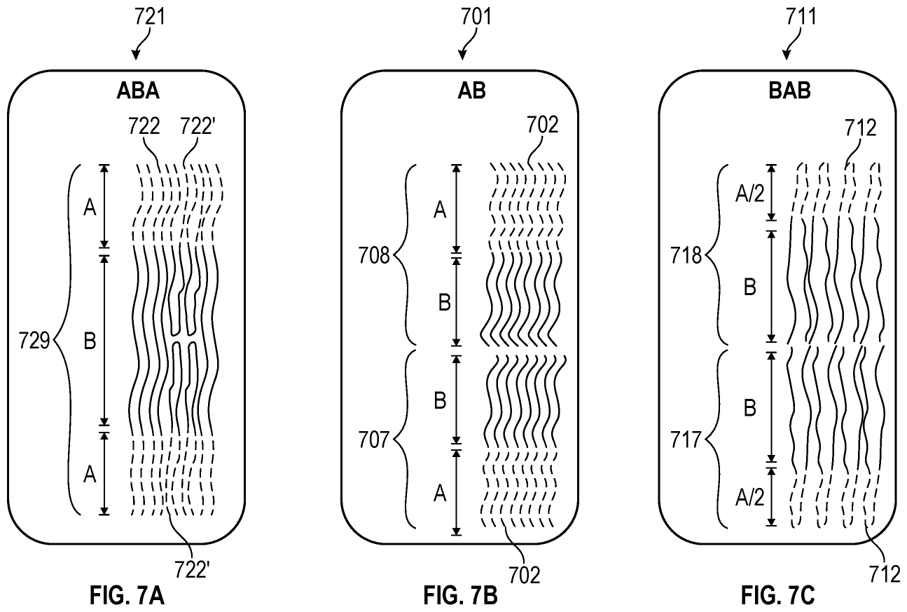

FIGS. 7A-7C schematically illustrate further details of barriers using polymers which may be included in the nanopore composition and device of FIG. 1 and used in barriers such as described with reference to FIGS. 2A-2B and 3-6. It will be appreciated that such barriers suitably may be adapted for use in any other composition or device, and are not limited to use with nanopores.

Referring now to FIG. 7A, barrier 721 uses a triblock “ABA” copolymer. Barrier 721 includes layer 729 which may include end groups 450 (not specifically illustrated) respectively contacting fluids 120 or 120′. Layer 729 includes a plurality of molecules 722 of a triblock ABA copolymer. As illustrated in FIG. 7A, each molecule 722 of the triblock copolymer includes first and second hydrophilic blocks, each denoted “A” and being approximately of length “A,” and a hydrophobic block disposed between the first and second hydrophilic blocks, denoted “B” and being approximately of length “B”. The hydrophilic A blocks at first ends of molecules 722 (the molecules forming layer 729) may be coupled to end groups 450 forming a first outer surface of the barrier 721, e.g., contacting fluid 120. The hydrophilic A blocks at second ends of molecules 722 may be coupled to end groups 450 forming a second outer surface of the barrier 721, e.g., contacting fluid 120′. The hydrophobic B blocks of the molecules 722 are within the barrier 711 in a manner such as illustrated in FIG. 7C.

In the illustrated example of FIG. 7B, the majority of molecules 722 within layer 729 may extend substantially linearly and in the same orientation as one another. Optionally, as illustrated in the example of FIG. 7A, some of the molecules 722′ may be folded at their B blocks, such that both of the hydrophilic A blocks of such molecules may contact the same fluid as one another. Accordingly, the example shown in FIG. 7A may be considered to be partially a single layer, and partially a bilayer. In other examples (not specifically illustrated), layer 729 may be entirely a single-layer or may be entirely a bilayer, e.g., as also described with reference to FIG. 4. Regardless of whether the membrane includes molecules 722 which extend substantially linearly and/or molecules 722′ which are folded, as illustrated in FIG. 7A, Accordingly, layer 729 may have a thickness of approximately 2A+B. In some examples, length A is about 1 RU to about 100 RU, e.g., about 2 RU to about 100 RU, or about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. It will be appreciated that any end groups that are coupled to the hydrophilic blocks contribute to the overall thickness of the barrier. Optionally, barrier 721 described with reference to FIG. 7A may be suspended across an aperture in a manner such as described with reference to FIGS. 2A-2B and 4.

Referring now to FIG. 7B, barrier 701 uses a diblock “AB” copolymer. Barrier 701 includes first layer 707 which may contact fluid 120 and second layer 708 which may contact fluid 120′ as described with reference to FIG. 1. First layer 707 includes a first plurality of molecules 702 of a diblock AB copolymer, and second layer 708 includes a second plurality of the molecules 702 of the diblock AB copolymer. As illustrated in FIG. 7B, each molecule 702 of the diblock copolymer includes a hydrophobic block, denoted “B” and being approximately of length “B,” coupled to a hydrophilic block, denoted “A” and being approximately of length “A”. The hydrophilic A blocks of the first plurality of molecules 702 (the molecules forming layer 707) may include end groups 250 (not specifically shown) forming a first outer surface of the barrier 701, e.g., contacting fluid 120. The hydrophilic A blocks of the second plurality of molecules 702 (the molecules forming layer 708) may include end groups 250 (not specifically shown) forming a second outer surface of the barrier 702, e.g., contacting fluid 120′. The respective ends of the hydrophobic B blocks of the first and second pluralities of molecules (optionally including end groups 260, not specifically shown) contact one another within the barrier 701 in a manner such as illustrated in FIG. 7B.

As illustrated in the example of FIG. 7B, substantially all of the molecules 702 within layer 707 may extend substantially linearly and in the same orientation as one another, and similarly substantially all of the molecules 702 within layer 708 may extend substantially linearly and in the same orientation as one another (which is opposite that of the orientation the molecules within layer 707). Accordingly, first and second layers 707, 708 each may have a thickness of approximately A+B, and barrier 701 may have a thickness of approximately 2A+2B. In some examples, length A is about 1 repeating unit (RU) to about 100 RU, e.g., about 2 RU to about 100 RU, or about 5 RU to about 40 RU, or about 10 RU to about 30 RU, or about 10 RU to about 20 RU, or about 20 RU to about 40 RU, or about 13 RU to about 44 RU, or about 30 RU to about 44 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU, or about 13 RU to about 44 RU, or about 30 RU to about 44 RU. It will be appreciated that any end groups that are coupled to the hydrophilic or hydrophobic blocks contribute to the overall thickness of the barrier. Optionally, barrier 701 described with reference to FIG. 7B may be suspended across an aperture in a manner such as described with reference to FIGS. 2A-2B and 3.

Referring now to FIG. 7C, barrier 711 uses a triblock “BAB” copolymer. Barrier 711 includes first layer 717 which may contact fluid 120 and second layer 718 which may contact fluid 120′ in a manner similar to that described with reference to FIG. 1. First layer 717 includes a first plurality of molecules 712 of a triblock copolymer, and second layer 718 includes a second plurality of the molecules 712 of the triblock copolymer. As illustrated in FIG. 7C, each molecule 712 of the triblock copolymer includes first and second hydrophobic blocks, each denoted “B” and being approximately of length “B” and optionally being coupled to an end group 660 (not specifically illustrated), and a hydrophilic block disposed between the first and second hydrophobic blocks, denoted “A” and being approximately of length “A”. The hydrophilic A blocks of the first plurality of molecules 712 (the molecules forming layer 717) form a first outer surface of the barrier 711, e.g., contact fluid 120. The hydrophilic A blocks of the second plurality of molecules 712 (the molecules forming layer 718) form a second outer surface of the barrier 711, e.g., contact fluid 120′. The respective ends of the hydrophobic B blocks (or end groups 660 coupled thereto, not specifically illustrated) of the first and second pluralities of molecules contact one another within the barrier 711 in a manner such as illustrated in FIG. 7C.

In the illustrated example of FIG. 7C, substantially all of the molecules 712 within layer 717 may extend in the same orientation as one another, and may be folded at the A block so that the A block can contact the fluid while the B blocks are interior to the barrier 711. Similarly, substantially all of the molecules 712 within layer 718 may extend in the same orientation as one another (which is opposite that of the orientation the molecules within layer 717), and may be folded at their A blocks so that the A blocks contact the fluid while the B blocks are interior to the barrier 711. Accordingly, first and second layers 717, 718 each may have a thickness of approximately A/2+B, and barrier 711 may have a thickness of approximately A+2B. In some examples, length A is about 1 RU to about 100 RU, or 2 RU to about 100 RU, e.g., about 10 RU to about 80 RU, or about 20 RU to about 50 RU, or about 50 RU to about 80 RU, or about 13 RU to about 44 RU, or about 30 RU to about 44 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU, or about 13 RU to about 44 RU, or about 30 RU to about 44 RU. It will be appreciated that any end groups that are coupled to the hydrophobic blocks contribute to the overall thickness of the barrier. Optionally, barrier 711 described with reference to FIG. 7C may be suspended across an aperture in a manner such as described with reference to FIGS. 2A-2B and 6.

Referring now to FIG. 5 described further above, the barrier may have a thickness of approximately B. In some examples, length B is about 2 RU to about 100 RU, or about 5 RU to about 100 RU, e.g., about 10 RU to about 80 RU, or about 20 RU to about 50 RU, or about 50 RU to about 80 RU, or about 13 RU to about 44 RU, or about 30 RU to about 44 RU. It will be appreciated that any ionic groups that are coupled to the hydrophobic blocks contribute to the overall thickness of the barrier. In this regard, the ionic groups may be considered to correspond to “A” but may have a relatively low number of RUs, e.g., about 1 to about 5 RU, or about 1 to about 2 RU, or about 1 RU.

It will be appreciated that the layers of the various barriers provided herein may be configured so as to have any suitable dimensions. Illustratively, to form barriers of similar dimension as one another:

A-B-A triblock copolymer (FIG. 7A) may have 2 hydrophilic blocks, each of length A (each A block is of M_{w}=x) and 1 hydrophobic block of length B (M_{w}=y); when self-assembled, those A-B-A triblock copolymers would form membranes with a top hydrophilic layer of length A, a core hydrophobic layer of length B, and a bottom hydrophilic layer of length A.

A-B diblock copolymer (FIG. 7B) may have 1 hydrophilic block of length A (M_{w}=x), and 1 hydrophobic block of length B (M_{w}=y/2); when self-assembled, those A-B diblock copolymers would form membranes with a top hydrophilic layer of length A, a core hydrophobic layer of length 2B, and a bottom hydrophilic layer of length A.

B-A-B triblock copolymer (FIG. 7C) may have 1 hydrophilic block of length A (M_{w}=x), and 2 hydrophobic blocks, each of length of B (each B block is of M_{w}=y/2); when self-assembled, those B-A-B triblock copolymers would form membranes with a top hydrophilic layer of length A/2, a core hydrophobic layer of length 2B, and a bottom hydrophilic layer of length A/2.

B block with ionic end groups (FIG. 5) may be considered to be an ABA block copolymer, where A corresponds to the ionic end group.

Additionally, or alternatively, the polymer packing into the layer(s) of the membrane may affect the hydrophilic ratio for each of the barriers, where hydrophilic ratio may be defined as the ratio between molecular mass of the hydrophilic block and the total molecular weight (MW or M_{w}) of the block copolymer (BCP) (hydrophilic ratio=M_{w} hydrophilic block/M_{w} BCP). For example:
A-B-A triblock copolymer (FIG. 7A or FIG. 5), hydrophilic ratio=2x/(2x+y);
A-B diblock copolymer (FIG. 7B), hydrophilic ratio=x/(x+y/2); and
B-A-B triblock copolymer (FIG. 7C), hydrophilic ratio=x/(x+y).

The present polymers may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Nonlimiting examples of zwitterionic monomers that may be polymerized to form zwitterionic polymers include:

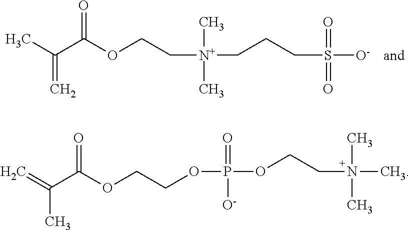

### Nonlimiting Examples of Hydrophilic Polypeptides Include:

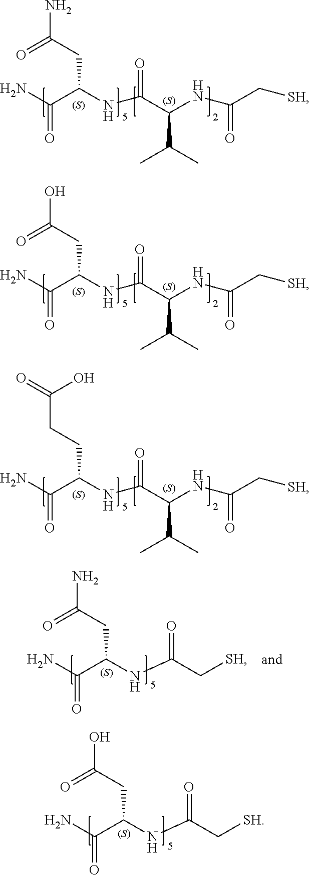

A nonlimiting example of a charged polyacrylamide is

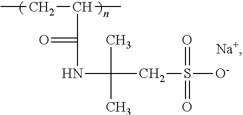

where n is between about 2 and about 100. Nonlimiting examples of nitrogen containing units include:

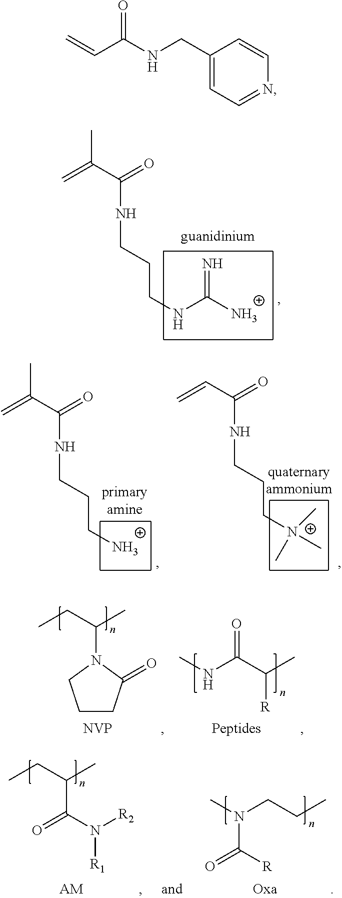

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Nonlimiting examples of hydrogenated polydienes include saturated polybutadiene (PBu), saturated polyisoprene (PI), saturated poly(myrcene),

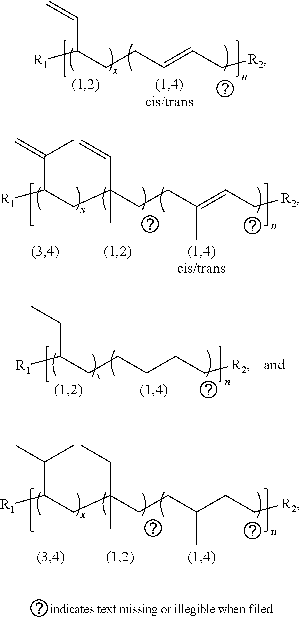

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen, and R₂ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. In other nonlimiting examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. A nonlimiting example of fluorinated polyethylene is

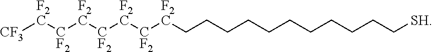 [SMILES: FC(F)(F)C(F)(F)C(F)(F)C(F)(F)C(F)(F)C(F)(F)C(F)(F)C(F)(F)CCCCCCCCCCCS]

Nonlimiting examples of hydrophobic polypeptides include (0<x<1):

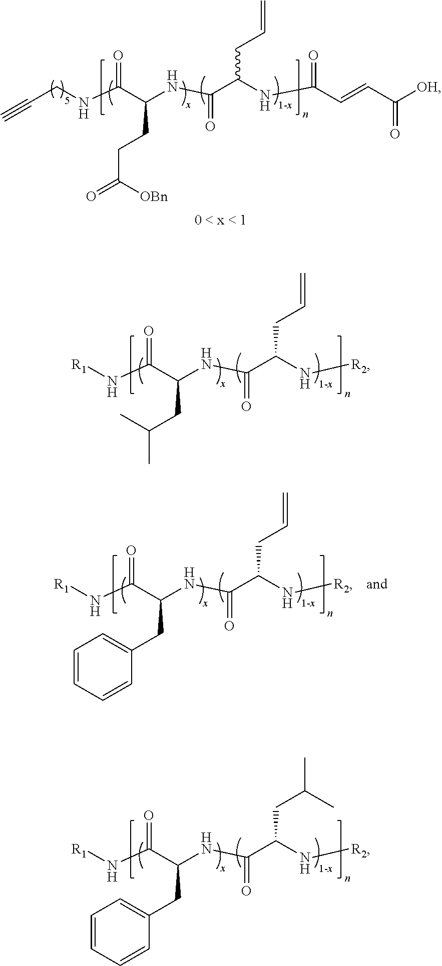

where n is between about 2 and about 100.

A variety of suitable end groups for coupling to the hydrophobic and/or hydrophilic blocks may be envisioned. Nonlimiting examples of end groups that may be coupled to the ends of hydrophilic blocks (e.g., in a manner such as described with reference to FIG. 2A, 3, or 4) include but are not limited to fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

A nonlimiting example of an end group that may be coupled to the end of a hydrophobic block (e.g., in a manner such as described with reference to FIG. 2A or 6) is a lower alkyl (C₁₋₄ alkyl) such a methyl, ethyl, propyl, or n-butyl group, or an aryl group, polycyclic aromatic hydrocarbon, or fluorinated alkyl or fluorinated aryl group. Nonlimiting examples of ionic end groups that may be coupled to hydrophobic polymers (e.g., in a manner such as described with reference to FIG. 5) are those that include zwitterions, those that include cations, and those that include anions. End groups that include zwitterions include, but are not limited to: 2-methacryloyloxyethyl phosphorylcholine, 3-[dimethyl-2-(2-methylprop-2-enoyloxy)ethyl]azaniumyl]propane-1-sulfonate (DMAPS), 3-{[3-(acryloylamino)propyl](dimethyl)ammonio}propanoate, and 3-{[3-(acryloylamino)propyl](dimethyl)ammonio}-1-propanesulfonate. The zwitterionic end group may be coupled to the polymer using any suitable type of linker, such as sulfide, ether, ester, alkyl, triazole, or the like.

Example polymers including ionic end groups are shown below:

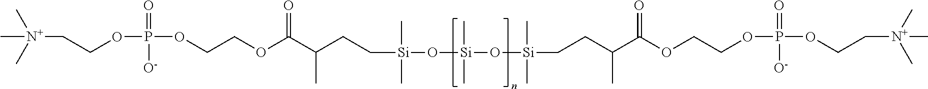

(PDMS-phosphorylcholine) where n is between about 2 and about 100,

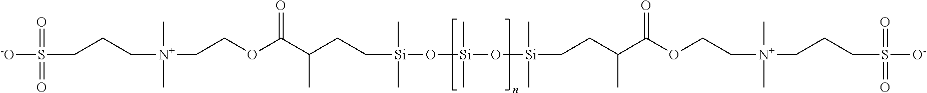

(PDMS ammonium sulfonate ester linker) where n is between about 2 and about 100,

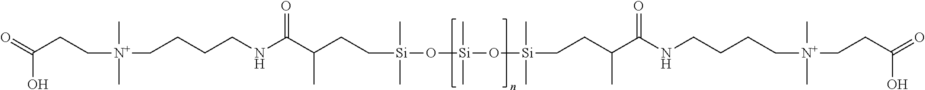

(PDMS ammonium carboxylate; note that the carboxylate may be substantially deprotonated at neutral pH, as follows:

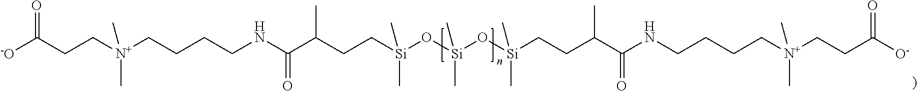

where n is between about 2 and about 100,

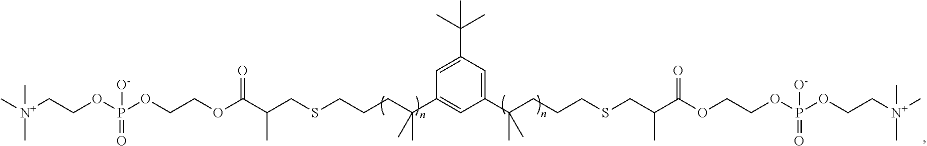

(PIB-phosphorylcholine), where n is between 2 and about 100, and

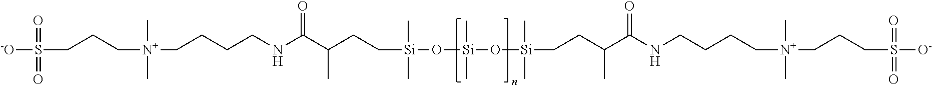

(PDMS ammonium sulfonate, amide linker) where n is between about 2 and about 100.

End groups that include cations include, but are not limited to, 2-(trimethylammonio)ethyl methacrylate. An example polymer including an end group with a cation is shown below:

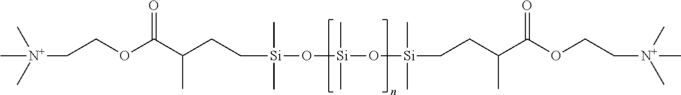

(trimethyl ammonium), where n is between about 2 and about 100.

End groups that include anions include, but are not limited to, 3-sulfopropyl acrylate, 2-propene-1-sulfonate, or vinylphosphonic acid. Example polymers including end groups with anions are shown below:

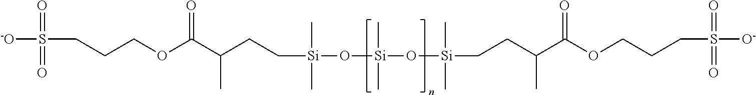

(PDMS propyl sulfonate), where n is between about 2 and about 100,

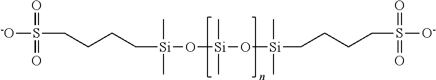

(PDMS sulfonate), where n is between about 2 and about 100, and

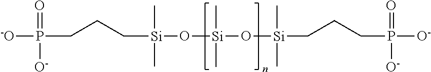

(PDMS phosphonic acid), where n is between about 2 and about 100.

End groups may be coupled to hydrophobic and/or hydrophilic blocks in any suitable manner. In some examples, an end group is coupled to a hydrophobic or hydrophilic block via an amide linkage or via the product of, for example but not limited to, hydrosilylation, amine-ester coupling, CuAAC click chemistry, DBCO-azide, a thiol-Michael addition, or a thiol-ene click reaction. Example reaction schemes are provided further below.

In one nonlimiting example, an AB diblock copolymer includes PDMS-b-PEO, where “-b-” denotes that the polymer is a block copolymer. In another nonlimiting example, an AB diblock copolymer includes PBd-b-PEO. In another nonlimiting example, an AB diblock copolymer includes PIB-b-PEO. In another nonlimiting example, a BAB triblock copolymer includes PDMS-b-PEO-b-PDMS. In another nonlimiting example, a BAB triblock copolymer includes PBd-b-PEO-b-PBd. In another nonlimiting example, a BAB triblock copolymer includes PIB-b-PEO-b-PIB. In another nonlimiting example, an ABA triblock copolymer includes PEO-b-PBd-b-PEO. In another nonlimiting example, an ABA triblock copolymer includes PEO-b-PDMS-b-PEO. In another nonlimiting example, an ABA triblock copolymer includes PEO-b-PIB-b-PEO. In some examples, a hydrophobic polymer includes PDMS or PIB.

Hydrophobic block(s) may be coupled to hydrophilic block(s) in any suitable manner, e.g., via respective amide bonds or via the products of polymerization reactions. It will be appreciated that any suitable hydrophilic block(s) may be used with any suitable hydrophobic block(s), and that any suitable end group(s) may be coupled to one or both terminal ends of the block copolymers. Additionally, in examples including two hydrophilic blocks, those blocks may be but need not necessarily include the same polymers as one another, and may but need not necessarily be coupled to the same end groups as one another. Similarly, in examples including two hydrophobic blocks, those blocks may be but need not necessarily include the same polymers as one another, and may but need not necessarily be coupled to the same end groups as one another.

The respective molecular weights, glass transition temperatures, and chemical structures of the hydrophobic and hydrophilic blocks, and the end groups respectively coupled to the hydrophobic and/or hydrophilic groups, suitably may be selected so as to provide the barrier with appropriate stability for use and ability to insert a nanopore. For example, the respective molecular weights of the hydrophobic and hydrophilic blocks may affect how thick each of the blocks (and thus layers of the barrier) are, and may influence stability as well as capacity to insert the nanopore, e.g., through electroporation, pipette pump cycle, or detergent assisted pore insertion. Additionally, or alternatively, the ratio of molecular weights of the hydrophilic and hydrophobic blocks may affect self-assembly of those blocks into the layers of the barrier. Additionally, or alternatively, the respective glass transition temperatures (T_{g}) of the hydrophobic and hydrophilic blocks may affect the lateral fluidity of the layers of the barrier; as such, in some examples it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and stability of those layers. Additionally, or alternatively, chemical structures of the end groups may affect the way the chains get packed into the layers, and stability of those layers.

In nonlimiting examples provided herein, a barrier 401 such as described with reference to FIG. 4 may include molecules 421 of an ABA triblock copolymer in which the first and second hydrophilic blocks 442 may include PEO or other suitable hydrophilic polymer such as polyacrylamide, a polyalcohol, a polypeptide, a polyoxazoline, or poly-N-vinylpyrrolidone. The hydrophilic blocks may have any suitable length. For example, the first and second hydrophilic blocks each may include between about 2 and about 12 PEO repeating units, illustrative between about 2 and about 12 PEO repeating units, e.g., between about 2 and about 4 PEO repeating units, or between about 3 and about 9 PEO repeating units, or between about 9 and about 12 PEO repeating units. Additionally, or alternatively, in some examples, the hydrophobic block 441 may include PDMS or PIB. The hydrophobic block may have any suitable length. For example, the hydrophobic block may include about 2 to about 100 PDMS repeating units, e.g., about 13 to about 44 PDMS repeating units, e.g., about 30 to about 44 PDMS repeating units. Or, for example, the hydrophobic block may include about 2 to about 100 PIB repeating units e.g., about 13 to about 44 PIB repeating units, e.g., about 30 to about 44 PIB repeating units. The hydrophobic block (e.g., PDMS or PIB) may be coupled to the first and second hydrophilic blocks (e.g., PEO) via respective amide, sulfide, ether, ester, alkyl, or triazole bonds. The first and second end groups independently may be selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻). The end groups may be coupled to the PEO (or other hydrophilic polymer) directly or via amide or other suitable linkages in a manner such as shown below:

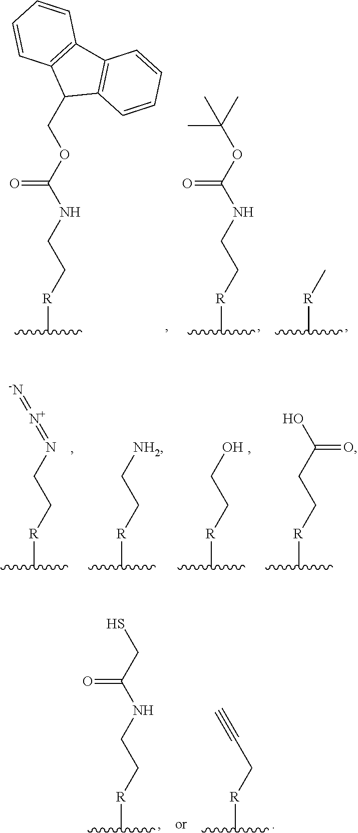

Note that although the illustrated structures include alkyl chains of specific lengths, other lengths suitably may be used, e.g., methylene or a two-carbon chain, and R may be 0, alkyl, or S in nonlimiting examples.

In one illustrative example, molecule 421 may include methyl end groups 250 and may have the following structure:

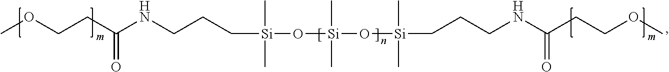

where m=about 2 to about 100, and n=about 2 to about 100.

A nonlimiting example of molecule 421, in which the end group is methyl and the number of PDMS and PEO RUs may be varied from that shown, is illustrated below:

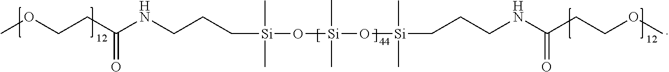

Another nonlimiting example of molecule 421, in which the end group is COOH and the number of PDMS and PEO RUs may be varied from that shown, is illustrated below:

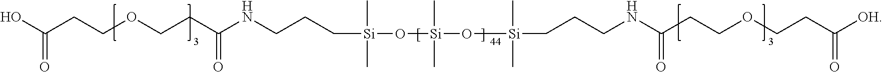

Another nonlimiting example of molecule 421, in which the end group is methyl and the number of PDMS and PEO RUs may be varied from that shown, is illustrated below:

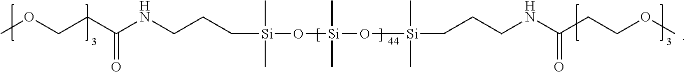

In other nonlimiting examples herein, a barrier 501 such as described with reference to FIG. 5 may include molecules 521 including first and second ionic end groups 550 and a hydrophobic block 541 that is disposed between and coupled to the first and second ionic end groups. The first and second ionic end groups 550 each may include a zwitterion, a cation, or an anion. Nonlimiting examples of end groups including zwitterions, cations, or anions are provided elsewhere herein. In one nonlimiting example, the hydrophobic block includes poly(dimethylsiloxane) (PDMS) or poly(isobutylene) (PIB). The hydrophobic block may have any suitable lengths. For example, the hydrophobic block may include about 2 to about 100 PDMS repeating units, e.g., about 13 to about 44 PDMS repeating units, e.g., about 30 to about 44 PDMS repeating units. Or, for example, the hydrophobic block may include about 2 to about 100 PIB repeating units e.g., about 13 to about 44 PIB repeating units, e.g., about 30 to about 44 PIB repeating units. The first and second ionic end groups may be coupled to the PDMS or PIB in any suitable manner, e.g., via the product of a hydrosilylation, amine-ester coupling, CuAAC click chemistry, DBCO-azide, a thiol-Michael addition, or a thiol-ene click reaction. A nonlimiting example of molecule 521, in which the end group includes a zwitterionic end group and the number of PDMS RUs may be varied from that shown, is illustrated below:

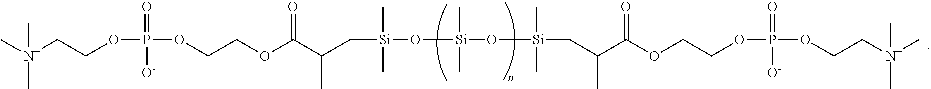

In still other nonlimiting examples herein, a barrier 301 such as described with reference to FIG. 3 may include molecules 221 of an AB diblock copolymer in which the hydrophilic block 232 may include PEO. The hydrophilic block may have any suitable length. For example, the hydrophilic block may include between about 2 and about 100 PEO repeating units, e.g., between about 2 and about 9 PEO repeating units. Additionally, or alternatively, the hydrophobic block 231 may include PDMS or PIB. The hydrophobic block may have any suitable length. For example, the hydrophobic block may include between about 2 and about 100 PDMS repeating units, e.g., between about 14 and about 44 PDMS repeating units, e.g., between about 14 and about 26 PDMS repeating units. Or, for example, the hydrophobic block may include between about 2 and about 100 PIB repeating units, e.g., between about 14 and about 44 PIB repeating units, e.g., between about 14 and about 26 PIB repeating units. In some examples, end group 250 may include fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), or sulfonate (SO₃⁻). In some examples, end group 260 may include a lower alkyl (C₁₋₄ alkyl) such as a methyl, ethyl, propyl, or n-butyl group, or an aryl group, polycyclic aromatic hydrocarbon, or fluorinated alkyl or fluorinated aryl group. A nonlimiting example of molecule 221 in which end group 250 is methyl and end group 260 is n-butyl is illustrated below:

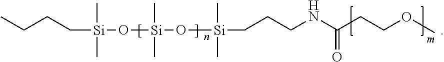

It will be appreciated that the number of PDMS, PIB, and/or PEO RUs suitably may be adjusted.

In further nonlimiting examples herein, a barrier 601 such as described with reference to FIG. 6 may include molecules 621 of a BAB triblock copolymer in which the hydrophilic block includes PEO. The hydrophilic block 642 may have any suitable length. For example, the hydrophilic block may include between about 7 and about 13 PEO repeating units. Additionally, or alternatively, the first and second hydrophobic blocks 641 may include PDMS or PIB. The hydrophobic blocks may have any suitable length. For example, the first and second hydrophobic blocks each may include between about 14 and about 26 PDMS repeating units. Or, for example, the first and second hydrophobic blocks may include between about 2 and about 100 PIB repeating units, e.g., between about 14 and about 44 PIB repeating units, e.g., between about 14 and about 26 PIB repeating units. End group 660 may include a lower alkyl (C₁₋₄ alkyl) such as a methyl, ethyl, propyl, or n-butyl group, or an aryl group, polycyclic aromatic hydrocarbon, or fluorinated alkyl or fluorinated aryl group. A nonlimiting example molecule 621 is illustrated below in which end groups 660 are propyl:

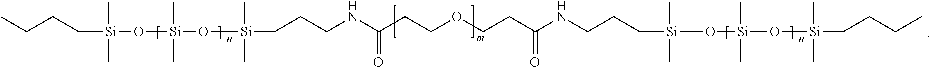

Although this particular example includes 4-carbon alkyl groups 660 at the terminal ends of the PDMS blocks, it will be appreciated that other end groups may be used such as described elsewhere herein. Additionally, it will be appreciated that the number of PDMS, PIB, and/or PEO RUs suitably may be adjusted.

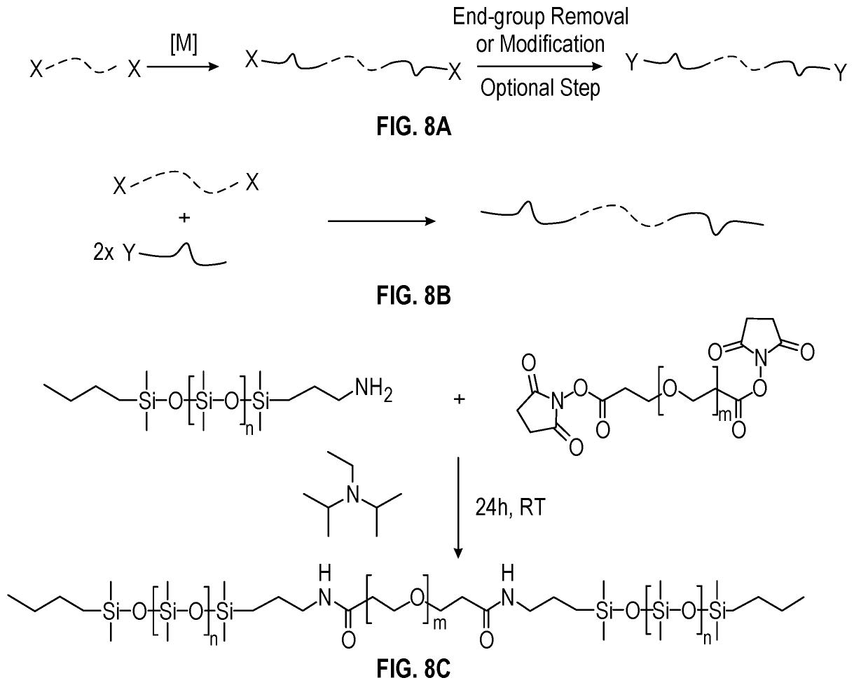

It will be appreciated that the present diblock and triblock copolymers may be made using any suitable combination of operations. FIGS. 8A-8C schematically illustrate example schemes for preparing triblock copolymers for use in the nanopore composition and device of FIG. 1. In some examples, the present diblock and triblock copolymers may be made using a “macro-initiator” approach such as illustrated in FIG. 8A in which one polymer block is made first and then used as an initiator (X in FIG. 8A) to grow one or more additional blocks using monomers ([M] in FIG. 8A). Illustratively, operations for making a diblock copolymer may include polymerizing a plurality of hydrophilic monomers to form a hydrophilic polymer; forming an initiator at a terminal end of the hydrophilic polymer; and using the initiator to polymerize a plurality of hydrophobic monomers to form a hydrophobic polymer coupled to the hydrophilic polymer. Alternatively, operations for making a diblock copolymer may include polymerizing a plurality of hydrophobic monomers to form a hydrophobic polymer; forming an initiator at a terminal end of the hydrophobic polymer; and using the initiator to polymerize a plurality of hydrophilic monomers to form a hydrophilic polymer coupled to the hydrophobic polymer. Similarly, operations for making a triblock BAB copolymer may include polymerizing a plurality of hydrophilic monomers to form a hydrophilic polymer; forming initiators at respective terminal ends of the hydrophilic polymer; and using the initiators to polymerize a plurality of hydrophobic monomers to form a hydrophobic polymer coupled to each terminal end of the hydrophilic polymer. Similarly, operations for making a triblock ABA copolymer may include polymerizing a plurality of hydrophobic monomers to form a hydrophobic polymer; forming initiators at respective terminal ends of the hydrophobic polymer; and using the initiators to polymerize a plurality of hydrophilic monomers to form a hydrophilic polymer coupled to each terminal end of the hydrophobic polymer. In such a “macro-initiator” approach, the initiator (X in FIG. 8A) suitably may be selected based on the particular monomers being used and the particular type of polymerization being performed. For example, for an atom transfer free radical polymerization (ATRP), the initiator may include bromine or chlorine. Or, for example, for a reversible addition fragmentation chain transfer (RAFT) polymerization, the initiator may include a chain transfer agent. After polymerization is complete, the end group(s) (X in FIG. 8A) may be modified or removed (e.g., to provide end group(s) Y in FIG. 8A).

In other examples, the present diblock and triblock copolymers may be made using a “coupling” approach such as illustrated in FIG. 8B in which polymer blocks are made separately and then coupled together using reactive moieties (X and Y in FIG. 8B). Illustratively, operations for making a diblock copolymer may include polymerizing a plurality of hydrophilic monomers to form a hydrophilic polymer; polymerizing a plurality of hydrophobic monomers to form a hydrophobic polymer; and coupling the hydrophilic polymer to the hydrophobic polymer. Operations for making a triblock copolymer may include polymerizing a plurality of hydrophilic monomers to form a hydrophilic polymer having terminal ends; polymerizing a plurality of hydrophobic monomers to form first and second hydrophobic polymers; and coupling the first and second hydrophobic polymers to respective terminal ends of the hydrophilic polymer. Alternatively, operations for making a triblock copolymer may include polymerizing a plurality of hydrophilic monomers to form first and second hydrophilic polymers; polymerizing a plurality of hydrophobic monomers to form a hydrophobic polymer having terminal ends; and coupling the first and second hydrophilic polymers to respective terminal ends of the hydrophobic polymer. In such a “coupling” approach, a terminal end of the hydrophobic polymer may include a first reactive moiety (Y in FIG. 8B), and a terminal end of the hydrophilic polymer may include a second reactive moiety (X in FIG. 8B) that reacts with the first reactive moiety to couple the hydrophilic polymer to the hydrophobic polymer. The reactive moieties (X and Y in FIG. 8B) suitably may be selected based on the particular polymers being coupled and the type of coupling being performed. For example, “Click” chemistry moieties may be used. Illustratively, one of the first and second reactive moieties may include an azide and the other of the first and second reactive moieties may include an alkyne; or one of the first and second reactive moieties may include a thiol and the other of the first and second reactive moieties may include an alkene; or one of the first and second reactive moieties may include a thiol and the other of the first and second reactive moieties comprises an alkyne. Or, for example, amide linkers may be formed. Illustratively, one of the first and second reactive moieties may include an amine and the other of the first and second reactive moieties may include N-hydroxysuccinimide (NHS).

FIG. 8C illustrates a nonlimiting example in which the hydrophobic polymer is PDMS having an amine (NH₂) group at one of its terminal ends and a 3-carbon alkyl group at its other terminal end, the hydrophilic polymer is PEO having NHS at its terminal ends, and the amine and NHS groups are reacted with one another in the presence of triisopropylamine to provide a BAB triblock copolymer. Although this particular example includes 3-carbon alkyl groups 660 at the terminal ends of the PDMS blocks, it will be appreciated that other end groups may be used such as described elsewhere herein.

Another nonlimiting example of forming a PEO-PDMS-PEO ABA triblock copolymer with methyl end groups is shown below. In this example, a PDMS-bis allyl is reacted by thiol-ene click chemistry with a PEG-thiol; for the reaction to proceed, it is carried out under inert atmosphere using degassed solvent (e.g., chloroform) and in the presence of a photoinitiator (e.g., irgacure2959), and under UV exposure for 5-30 min.

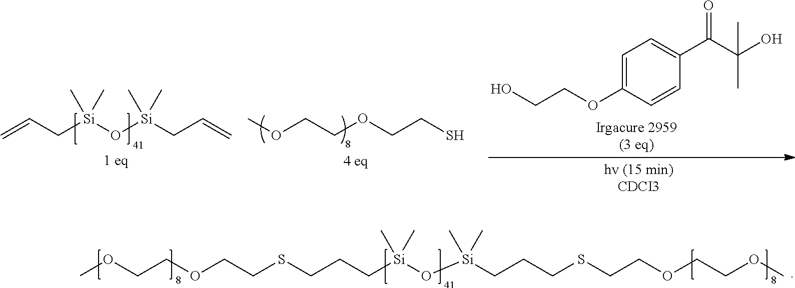

Another nonlimiting example of forming a triblock copolymer is shown below. In this example, a PIB-bis allyl is reacted by thiol-ene click chemistry with a PEG-thiol; for the reaction to proceed, it is carried out under inert atmosphere (dry Argon or dry Nitrogen) using degassed solvent (e.g., chloroform) and in the presence of a photoinitiator (e.g., irgacure 2959), and under UV exposure for 5-60 min. In some examples, the reaction is carried out under UV exposure for 2-180 min. with a UV power ranging from 1 mW/cm² to 100 mW/cm². In some examples, the UV wavelength used is 365 nm.

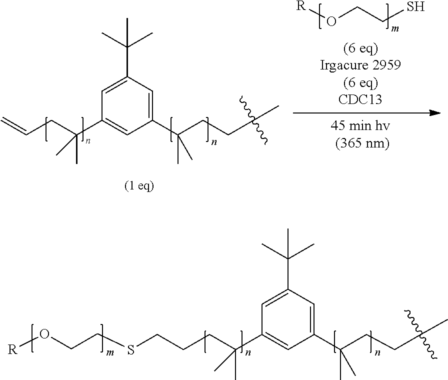

For nanopore sequencing applications, membrane fluidity can be considered beneficial. Without wishing to be bound by any theory, the fluidity of a block copolymer membrane is believed to be largely imparted by the physical property of the hydrophobic “B” blocks. More specifically, B blocks including “low T_{g}” hydrophobic polymers (e.g., having a T_{g} below around 0° C.) may be used to generate membranes that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

Hydrophobic B blocks with a low T_{g} may be used to help maintain membrane flexibility under conditions suitable for performing nanopore sequencing, e.g., in a manner such as described with reference to FIG. 9-12 or 17. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about −45° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C..

Hydrophobic B blocks with a fully saturated carbon backbone, such as PIB, also may be expected to increase chemical stability of the block copolymer membrane. Additionally, or alternatively, branched structures within the hydrophobic B block, such as with PIB, may be expected to induce chain entanglement, which may be expected to enhance the stability of the block copolymer membrane. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIG. 9-12 or 17).

In some examples of the AB copolymer shown below including PBd as the B block and PEO as the A block, R is selected from the group consisting of fluorenylmethoxycarbonyl (Fmoc), tert-butyl carbamate (NHBoc), methyl (CH₃), carboxyl (COOH), propargyl, azide (N₃), amino (NH₂), hydroxyl (OH), thiol (SH), biotin, or sulfonate (SO₃⁻); m=about 2 to about 100; and n=about 2 to about 100.

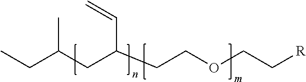

In some nonlimiting examples, R=OH; n=about 8 to about 50; and m=about 1 to about 20. In some nonlimiting examples, R=OH; n=about 10 to about 15; and m=about 5 to about 15.

In some examples of the ABA copolymer shown below including one or more PIB blocks as the B block and PEO as the A block, R₁ and R₂ are independently moieties selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻); V is an optional group that corresponds to a bis-functional initiator from which the isobutylene may be propagated and can be tert-butylbenzene, a phenyl connected to the hydrophobic blocks via the para, meta, or ortho positions, naphthalene, another aromatic group, an alkane chain with between about 2 and about 20 carbons, or another aliphatic group; m=about 2 to about 100; and n=about 2 to about 100. V may optionally be flanked by functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. When V is absent, only one PIB block is present and n=about 2 to about 100. L₁ and L₂ are independently linkers, which may include at least one moiety selected from the group consisting of an amide, a thioether (sulfide), a succinic group, a maleic group, an alkyl group (e.g., a methylene), an ether, and a product of a “click” reaction.

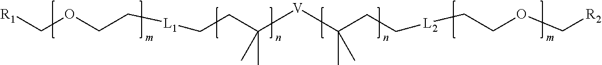

In some nonlimiting examples of the above structure, n=about 2 to about 50, and m=about 1 to about 50, R₁=R₂=COOH, V=tert-butylbenzene, and L₁=L₂=ethyl sulfide. In other nonlimiting examples, n=about 5 to about 20, m=about 2 to about 15, R₁=R₂=COOH, V=tert-butylbenzene, and L₁=L₂=ethyl sulfide. In other nonlimiting examples, n=about 13 to about 19, m=about 2 to about 5, R₁=R₂=COOH, V=tert-butylbenzene, and L₁=L₂=ethyl sulfide. In other nonlimiting examples, n=about 7 to about 13, m=about 7 to about 13, R₁=R₂=COOH, V=tert-butylbenzene, and L₁=L₂=ethyl sulfide. In particular, in one nonlimiting example (the structure of which is shown below), n=16, m=3, R₁=R₂=COOH, V=tert-butylbenzene, and L₁=L₂=ethyl sulfide. In another nonlimiting example (the structure of which is shown below, n=10, m=10, and R₁=R₂=COOH, V=tert-butylbenzene, and L₁=L₂=ethyl sulfide. In another nonlimiting example (the structure of which is shown below), n=16, m=8, R₁=R₂=CH₃, V=tert-butylbenzene, and L₁=L₂=ethyl sulfide.

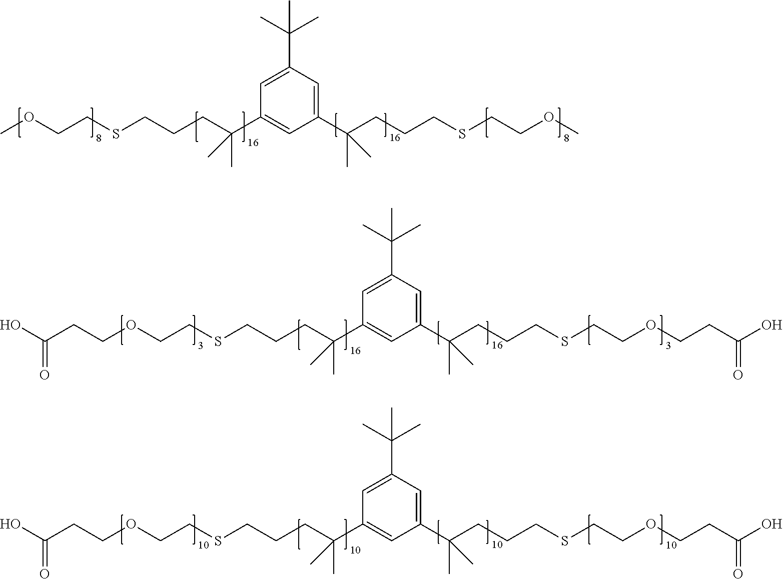

In another nonlimiting example of the more general structure shown above, the end groups may be zwitterionic, e.g., as shown below:

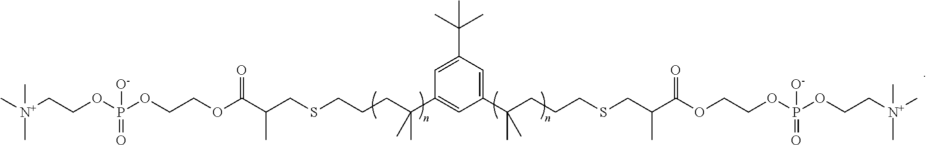

In some examples, multifunctional precursors may be sourced and used as precursors to the synthesis of bifunctional initiators to which V corresponds in the example further above. For example, the multifunctional precursor may be 5-tert-butylisophthalic acid (TBIPA) which can be synthesized into 1-(tert-butyl)-3,5-bis(2-methoxypropan-2-yl)benzene (TBDMPB) using reactions known in the art. In another example, TBIPA may be synthesized into 1-tert-butyl-3,5-bis(2-chloropropan-2-yl)benzene using reactions known in the art. The use of such bifunctional initiators allows cationic polymerization on both sides of the initiator, generating bifunctional PIBs, such as allyl-PIB-allyl, which can then be coupled to hydrophilic A blocks to generate ABA block copolymers including PIB as the B block. Here, although the bifunctional initiator may be located between first and second PIB polymers, it should be understood that the first and second PIB polymers and the bifunctional initiator (V) together may be considered to form a B block, e.g., of an ABA triblock copolymer.

In another nonlimiting example, an ABA triblock copolymer includes

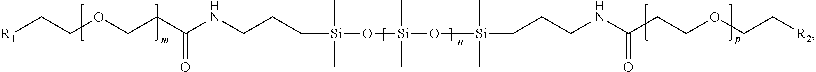

where m=about 2 to about 100, n=about 2 to about 100, p=about 2 to about 100, R₁ and R₂ are independently selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻). In some nonlimiting examples, m=about 2 to about 30, n=about 25 to about 45, p=about 2 to about 30, R₁ and R₂ are independently selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻). In some nonlimiting examples, m=about 2 to about 15, n=about 30 to about 40, p=about 2 to about 15, R₁ and R₂ are independently selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻). In some nonlimiting examples, m=about 7 to about 11, n=about 35 to about 40, p=about 7 to about 11, R₁ and R₂ are independently selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻). In some nonlimiting examples, m=about 2 to about 5, n=about 30 to about 37, p=about 2 to about 5, R₁ and R₂ are independently selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

In particular, as shown below, in one nonlimiting example, m=3, n=34, p=3, and R₁=R₂=COOH. In another nonlimiting example shown below, m=9, n=37, p=9, and R₁=R₂=COOH.

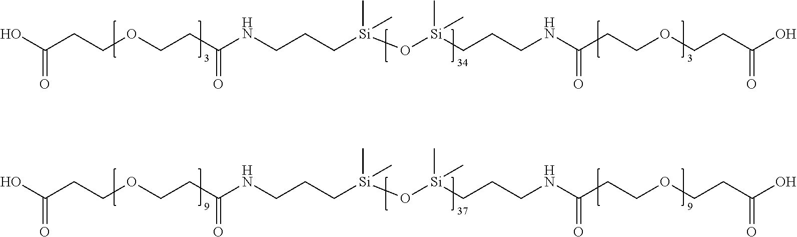

In some examples of the AB copolymer shown below including a PIB block as the B block and PEO as the A block, R is a moiety selected from the group consisting of fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻); m=about 2 to about 100; n=about 2 to about 100; and L is a linker selected from the group consisting of an amide, a thioether (sulfide), a succinic group, a maleic group, an alkyl group (e.g., methylene), an ether, or a product of a click reaction.

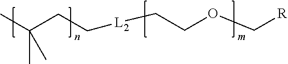

In particular, as shown below, in one nonlimiting example, n=13, m=8, R is a methyl, and L is ethyl sulfide. In another nonlimiting example shown below, n=13, m=3, R is a carboxyl group, and L is ethyl sulfide. In another nonlimiting example shown below, n=30, m=8, R is a methyl, and L is ethyl sulfide. In another nonlimiting example shown below, n=30, m=3, R is a carboxyl group, and L is ethyl sulfide.

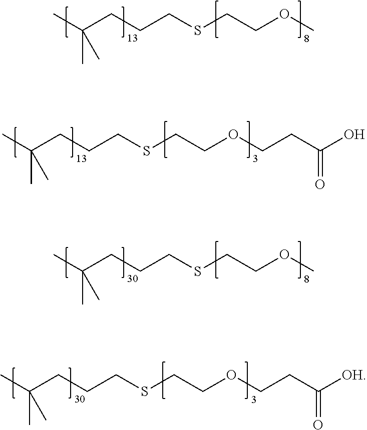

The scheme below shows an example synthetic route for an ionic (e.g., zwitterionic) functionalized PDMS via hydroxylation, wherein a zwitterionic monomer (here, a zwitterionic monomer including both a phosphoric acid and quaternary ammonium chloride moiety) that has an acrylate, methacrylate (as here), acrylamide, or methacrylamide functionality can be reacted with a hydride terminated PDMS:

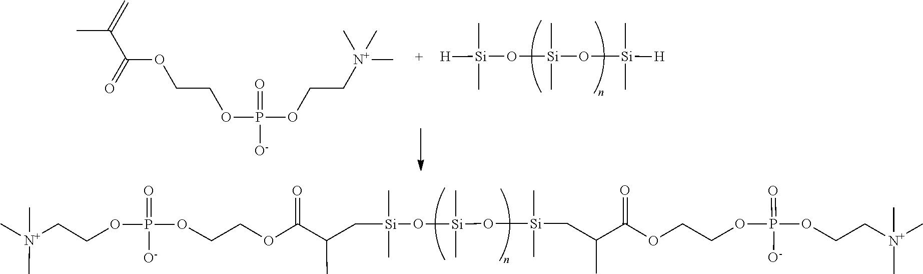

The reaction may be performed in the presence of a platinum catalyst (Karstedts) and a solvent or cosolvent mixture in which both PDMS and the zwitterionic monomer are soluble.

The scheme below shows an example synthetic route for zwitterionic functionalized PDMS via thiolation, wherein a thiol-functionalized zwitterionic monomer (here, SH-terminated 2-methacryloyloxyethyl phosphorylcholine-derivative) is reacted with an allyl functionalized PDMS in a thio-ene click reaction:

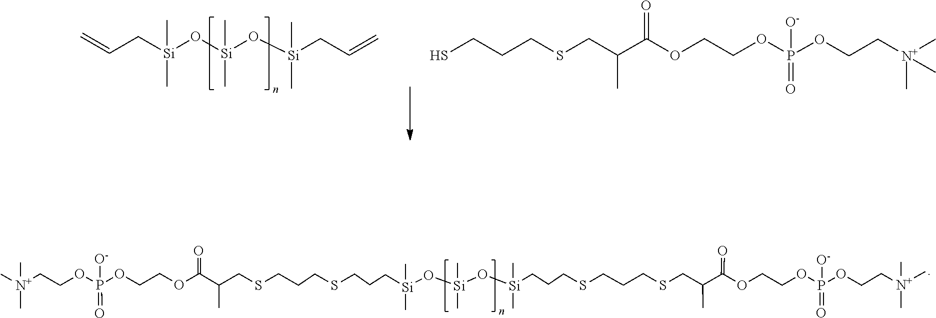

Ionic monomers (e.g., zwitterionic, anionic, or cationic monomers) that have acrylate, methacrylate, acrylamide, or methacrylamide functionalities (such as useful in a hydroxylation reaction) may be functionalized to include thiol functionality (such as useful in a thiol-ene click reaction) using a thiol-Michael addition in the presence of a secondary base. For example, the scheme below shows the reaction of 1,4 butane di-thiol with the zwitterion 2-methacryloyloxyethyl phosphorylcholine:

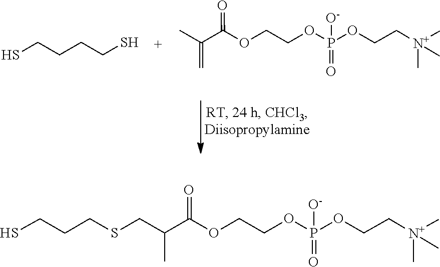 [SMILES: SCCCCS ; C=C(C)C(=O)OCCOP(=O)([O-])OCC[N+](C)(C)C ; CC(CSCCCS)C(=O)OCCOP(=O)([O-])OCC[N+](C)(C)C]

It will be appreciated that any suitable dithiol can be used to couple a thiol-terminated linker to an ionic monomer having an acrylate, methacrylate, acrylamide, or methacrylamide functionality via a thiol-Michael addition in a manner such as illustrated in the above scheme.

In some examples, the membrane may have a survival rate of 70% or more, 80% or more, 90% or more, or 95% or more when subjected to a voltage of 450 mV across the barrier. In some examples, the membrane has an open pore current at 100 mV of 95 pA or more, or 100 pA or more. In some examples, the membrane has an open pore current at 50 mV of 32 pA or more, 34 pA or more, or 36 pA or more. In some examples, the membrane-pore RMS noise is 2.2 pA or less, 2.0 pA or less, 1.8 pA or less, 1.6 pA or less, or 1.5 pA or less. In some examples, the membrane has a signal-to-noise ratio of 40 or more, 50 or more, 60 or more, or 70 or more. In some examples, the membrane has a membrane painting yield of 90% or more, or 95% or more.

In some examples, the membrane has a pore insertion voltage between about 300 mV and about 1100 mV. In some examples, the membrane has a single pore percentage after insertion of 85% or more, 90% or more, or 95% or more. In some examples, the membrane has a single pore survival rate of 90% or more, or 95% or more. In some examples, the membrane has a single pore current standard deviation of 2 pA or less, 1 pA or less, or 0.5 pA or less.

In some examples, a waveform is applied to the barrier which is made of a train of positive voltage micro pulses, spaced by negative voltage periods at −100 mV for 100 ms. The train of positive voltage pulses has a total of 20 pulses, with duration of 10 μs. The spacings between them have a set duration value of 30 ms and a voltage held at +50 mV. During a first cycle, the waveform may be applied continuously for a period of 5 minutes and the magnitude of the pulses kept at +700 mV. In further applied cycles (applied again for 5 mins each), the pulsing intensity is increased from +700 mV to +1200 mV in 100 mV steps, for a total of six cycles. In some examples, the membrane survival rate under such a waveform is 60% or more, 80% or more, 90% or more, or 95% or more. In some examples, the voltage at 50% membrane survival is 1000 mV or more, or 1200 mV or pore. In some examples, the voltage at 50% membrane and single pore survival is 900 mV or more, or 1000 mV or more.

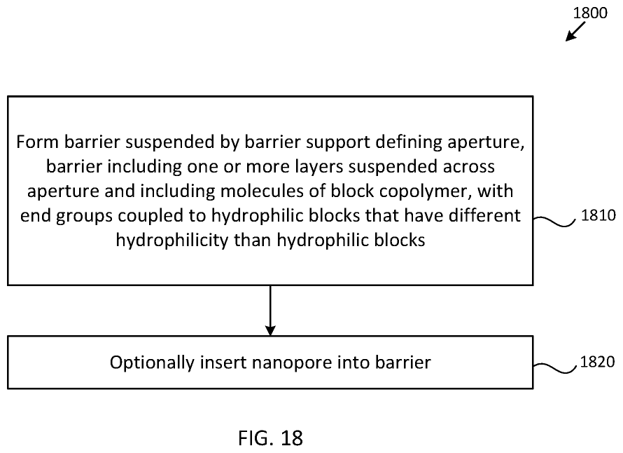

It will be appreciated that compositions and devices such as described with reference to FIGS. 1-12 and 17 may be prepared in any suitable manner. FIG. 18 illustrates a flow of operations for forming a device such as illustrated in FIG. 1. Method 1800 illustrated in FIG. 18 includes forming a barrier between first and second fluids, the barrier being suspended by a barrier support defining an aperture, the barrier including one or more layers suspended across the aperture and comprising molecules of a block copolymer, with end groups coupled to hydrophilic blocks that have a different have a different hydrophilicity than the hydrophilic blocks (operation 1810). The barrier may be formed using any suitable combination of operations provided herein or otherwise known in the art. For example, forming the barrier may include “painting” as known in the art. Known techniques for painting barriers that are suspended by barrier supports include brush painting (manual), mechanical painting (e.g., using stirring bar), and bubble painting (e.g., using flow through the device).

Each molecule of the block copolymer may include one or more hydrophilic blocks having an approximate length A and one or more hydrophobic blocks having an approximate length B. The one or more hydrophilic blocks may form outer surfaces of the barrier and the hydrophobic blocks being located within the barrier. For example, the barrier may include any AB or ABA copolymer provided herein. In some examples, the one or more hydrophobic blocks may include a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). For reasons such as explained elsewhere herein, such polymers may be expected to generate suspended membranes with particularly useful qualities for use in nanopore sequencing operations, e.g., such as described with reference to FIGS. 9-12 and 17. Any suitable end groups may be coupled to the hydrophilic blocks. Illustratively, the end groups may be selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

Method 1800 optionally also includes inserting a nanopore into the barrier (operation 1820). The nanopore may provide contact between the first fluid and the second fluid. The nanopore may be inserted into the barrier using operations such as described elsewhere herein, or otherwise known in the art. Known techniques for inserting a nanopore into a suspended barrier include electroporation, pipette pump cycle, and detergent assisted pore insertion. Tools for forming suspended barriers using synthetic polymers and inserting nanopores in the suspended barriers are commercially available, such as the Orbit 16 TC platform available from Nanion Technologies Inc. (California, USA). It will be appreciated that operation 820 need not necessarily be performed after operation 810, if it is desired to use the barrier without a nanopore.

Illustratively, the block copolymer in FIG. 18 is an AB diblock copolymer, such as described with reference to FIGS. 2A-2B, and 3. As such, the barrier may have a thickness of approximately 2A+2B. In one nonlimiting example of such a diblock copolymer, the hydrophobic block may be polybutadiene (PBd). Alternatively, the block copolymer may be an ABA triblock copolymer having two hydrophilic blocks and one hydrophobic block. As such, the barrier may have a thickness of approximately 2A+B. In one nonlimiting example of such a triblock copolymer, the hydrophobic block is poly(isobutylene) (PIB) or PDMS.

In other examples not specifically illustrated in FIG. 18, but contemplated herein, the block copolymer may be a BAB triblock copolymer having two hydrophobic blocks and one hydrophilic block, where the end groups are coupled to the hydrophobic blocks in a manner such as described with reference to FIG. 6. As such, the barrier may have a thickness of approximately A+2B. In still other examples not specifically illustrated in FIG. 18, but contemplated herein, the block copolymer may include a hydrophobic block coupled to first and second ionic groups. As such, the barrier may have a thickness of approximately B.

Devices and Methods Using Barriers for Nanopore Sequencing

It will further be appreciated that the present barriers may be used in any suitable device or application. For example, FIG. 9 schematically illustrates a cross-sectional view of an example use of the composition and device of FIG. 1. Device 900 illustrated in FIG. 9 may be configured may include fluidic well 100′, barrier 901 which may have a configuration such as described with reference to FIG. 2A-2B, 3, 4, 5, 6, or 7A-7C, first and second fluids 120, 120′, and nanopore 110 in a manner such as described with reference to FIG. 1. In the nonlimiting example illustrated in FIG. 9, second fluid 120′ optionally may include a plurality of each of nucleotides 921, 922, 923, 924, e.g., G, T, A, and C, respectively. Each of the nucleotides 921, 922, 923, 924 in second fluid 120′optionally may be coupled to a respective label 931, 932, 933, 934 coupled to the nucleotide via an elongated body (elongated body not specifically labeled). Optionally, device 900 further may include polymerase 905. As illustrated in FIG. 9, polymerase 905 may be within the second composition of second fluid 120′. Alternatively, polymerase 905 may be coupled to nanopore 110 or to barrier 901, e.g., via a suitable elongated body (not specifically illustrated). Device 900 optionally further may include first and second polynucleotides 940, 950 in a manner such as illustrated in FIG. 9. Polymerase 905 may be for sequentially adding nucleotides of the plurality to the first polynucleotide 940 using a sequence of the second polynucleotide 950. For example, at the particular time illustrated in FIG. 9, polymerase 905 incorporates nucleotide 922 (T) into first polynucleotide 940, which is hybridized to second polynucleotide 950 to form a duplex. At other times (not specifically illustrated), polymerase 905 sequentially may incorporate other of nucleotides 921, 922, 923, 924 into first polynucleotide 940 using the sequence of second polynucleotide 950.

Circuitry 180 illustrated in FIG. 9 may be configured to detect changes in an electrical characteristic of the aperture responsive to the polymerase sequentially adding nucleotides of the plurality to the first polynucleotide 940 using a sequence of the second polynucleotide 950. In the nonlimiting example illustrated in FIG. 9, nanopore 110 may be coupled to permanent tether 910 which may include head region 911, tail region 912, elongated body 913, reporter region 914 (e.g., an abasic nucleotide), and moiety 915. Head region 911 of tether 910 is coupled to nanopore 910 via any suitable chemical bond, protein-protein interaction, or any other suitable attachment that is normally irreversible. Head region 911 can be attached to any suitable portion of nanopore 910 that places reporter region 914 within aperture 913 and places moiety 915 sufficiently close to polymerase 905 so as to interact with respective labels 931, 932, 933, 934 of nucleotides 921, 922, 923, 924 that are acted upon by polymerase 905. Moiety 915 respectively may interact with labels 931, 932, 933, 934 in such a manner as to move reporter region 914 within aperture 913 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

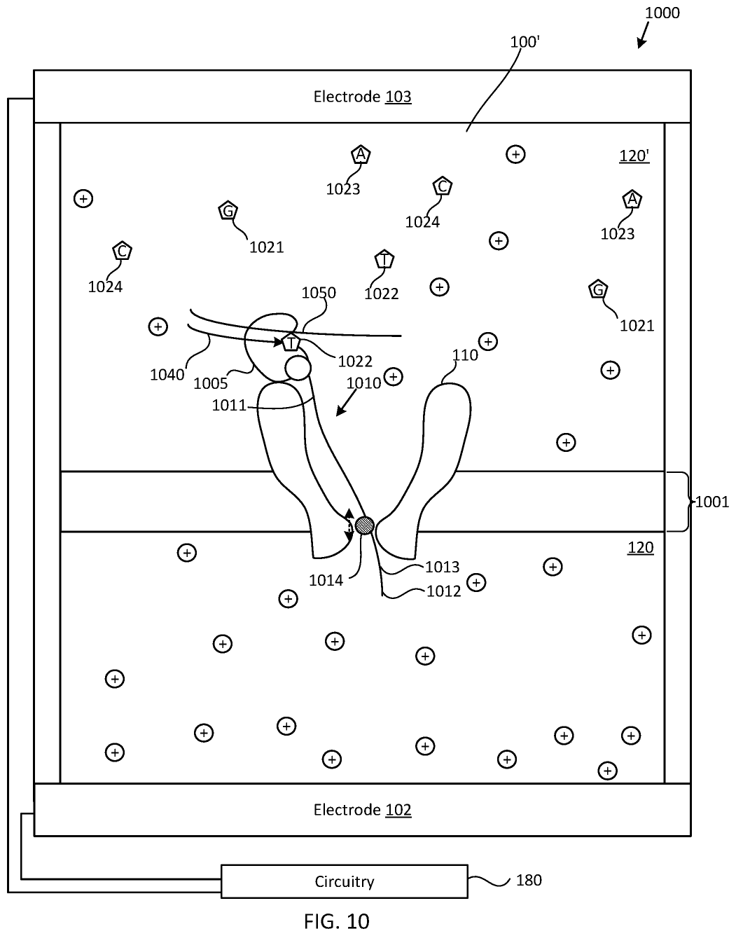

FIG. 10 schematically illustrates a cross-sectional view of another example use of the composition and device of FIG. 1. As illustrated in FIG. 10, device 1000 may include fluidic well 100′, barrier 1001 which may have a configuration such as described with reference to FIG. 2A-2B, 3, 4, 5, 6, or 7A-7C, first and second fluids 120, 120′, nanopore 110, and first and second polynucleotides 1040, 1050, all of which may be configured similarly as described with reference to FIG. 9. In the nonlimiting example illustrated in FIG. 10, nucleotides 1021, 1022, 1023, 1024 need not necessarily be coupled to respective labels. Polymerase 1005 may be coupled to nanopore 110 and may be coupled to permanent tether 1010 which may include head region 1011, tail region 1012, elongated body 1013, and reporter region 1014 (e.g., an abasic nucleotide. Head region 1011 of tether 1010 is coupled to polymerase 1005 via any suitable chemical bond, protein-protein interaction, or any other suitable attachment that is normally irreversible. Head region 1011 can be attached to any suitable portion of polymerase 1005 that places reporter region 1014 within aperture 113. As polymerase 1005 interacts with nucleotides 1021, 1022, 1023, 1024, such interactions may cause polymerase 1005 to undergo conformational changes. Such conformational changes may move reporter region 1014 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

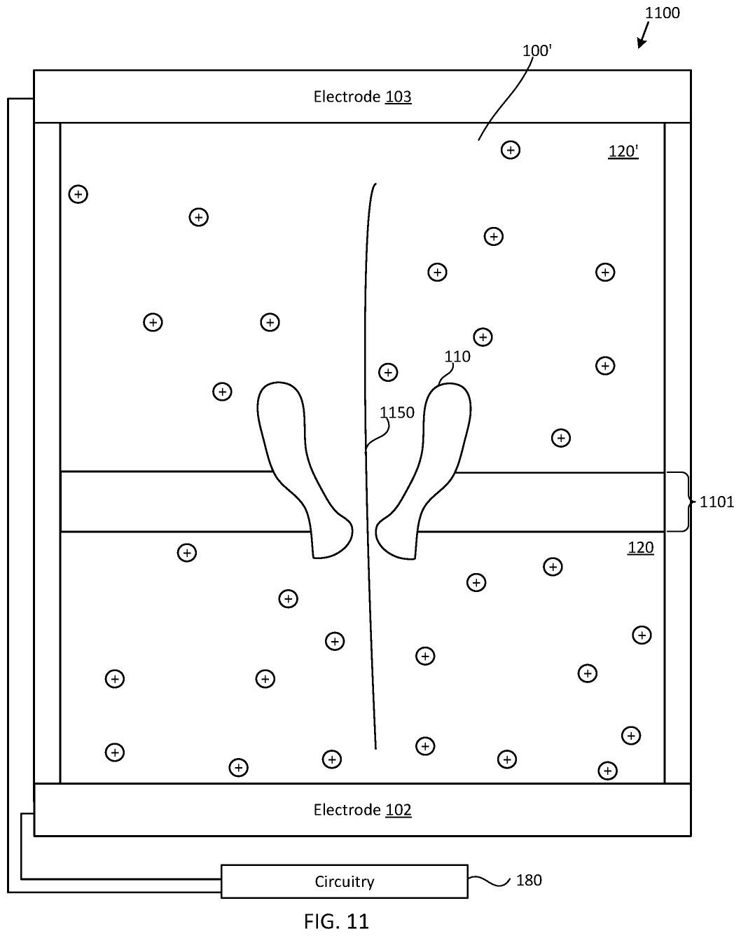

FIG. 11 schematically illustrates a cross-sectional view of another example use of the composition and device of FIG. 1. As illustrated in FIG. 11, device 1100 may include fluidic well 100′, barrier 1101 which may have a configuration such as described with reference to FIG. 2A-2B, 3, 4, 5, 6, or 7A-7C, first and second fluids 120, 120′, and nanopore 110 all of which may be configured similarly as described with reference to FIG. 9. In the nonlimiting example illustrated in FIG. 11, polynucleotide 1150 is translocated through nanopore 110 under an applied force, e.g., a bias voltage that circuitry 180 applies between electrode 102 and electrode 103. As bases in polynucleotide 1150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein.

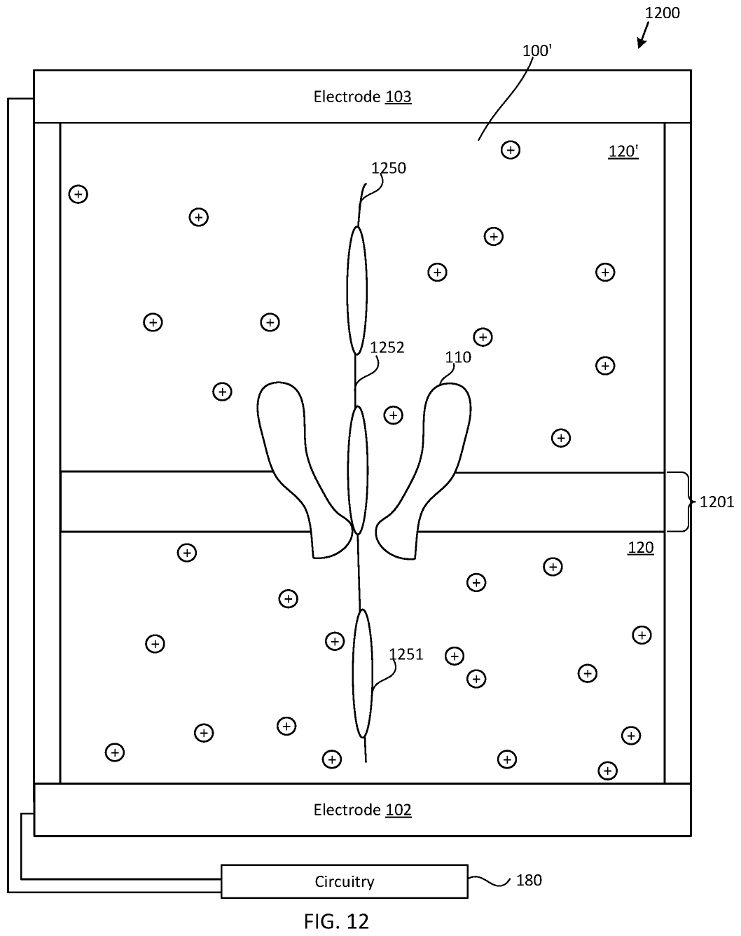

FIG. 12 schematically illustrates a cross-sectional view of another example use of the composition and device of FIG. 1. As illustrated in FIG. 12, device 1200 may include fluidic well 100′, barrier 1201 which may have a configuration such as described with reference to FIG. 2A-2B, 3, 4, 5, 6, or 7A-7C, first and second fluids 120, 120′, and nanopore 110 all of which may be configured similarly as described with reference to FIG. 9. In the nonlimiting example illustrated in FIG. 12, surrogate polymer 1250 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. In the example illustrated in FIG. 12, surrogate polymer 1250 includes labels 1251 coupled to one another via linkers 1252. An XPANDOMER™ is a particular type of surrogate polymer developed by Roche Sequencing, Inc. (Pleasanton, CA). XPANDOMERS™ may be prepared using Sequencing By eXpansion™ (SBX™, Roche Sequencing, Pleasanton CA). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANDOMERS™, linkers (tethers), labels, engineered polymerases, and methods for SBX™ see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

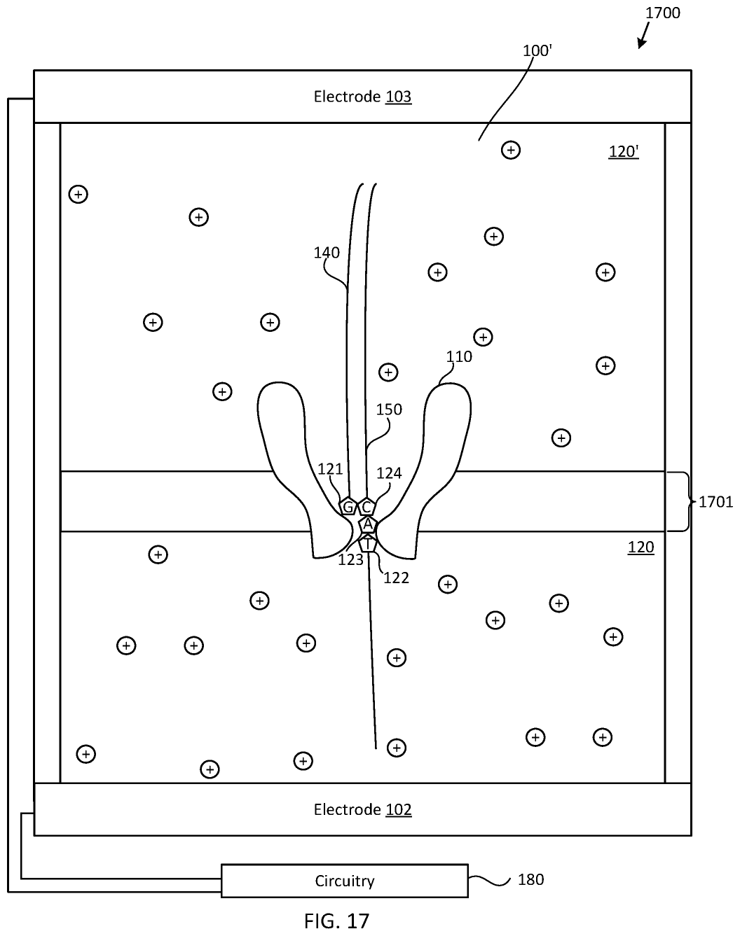

FIG. 17 schematically illustrates a cross-sectional view of another example use of the composition and device of FIG. 1. As illustrated in FIG. 17, device 100 may include fluidic well 100′, barrier 1701 which may have a configuration such as described with reference to FIGS. FIG. 2A-2B, 3, 4, 5, 6, or 7A-7C, first and second fluids 120, 120′, and nanopore 110 all of which may be configured similarly as described with reference to FIG. 9. In the nonlimiting example illustrated in FIG. 17, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see US Patent Publication No. 2023/0090867 to Mandell et al., the entire contents of which are incorporated by reference herein.

### WORKING EXAMPLES

The following examples are intended to be purely illustrative, and not limiting of the present invention unless otherwise recited in the claims.

### Example 1

Suspended barriers were prepared using supports with circular apertures, using different materials as described below. All materials were tested on the Orbit-16 instrument from NanION. This tool allows mechanical painting by rotation of a Teflon stirring bar on top of the chip cavities as well as electrical testing of the membrane/pore construct (membrane capacitance measurement, nanopore I/V curve). The barriers were generated under standard buffer conditions (1M KCl, 50 mM HEPES, pH=7.4), and the materials were diluted in octane (5 mg/mL) prior to testing through membrane formation (also called membrane painting). FIG. 13 illustrates the voltage breakdown waveform used to assess barrier stability. Membrane stability was quantified as the percentage of membranes remaining at the end of each step of the voltage ramp illustrated. The voltage ramp was stepped in 50 mV steps from 150 mV to 500 mV, as shown in FIG. 13. Each step lasted for 10 seconds. Nanopore insertion was represented as the number of successful single nanopore insertions during each individual experiment with a maximum of 16 nanopores per experiment.

Barriers were formed using the following ABA polymer with methyl end groups (ABA1):

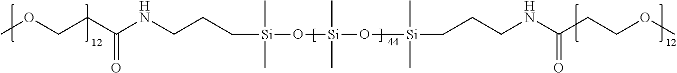

Reference barriers were formed using the phospholipid dipalmitoylphosphatidylcholine (DPhPC), which is widely used to form lipid bilayers, the structure of which is shown below:

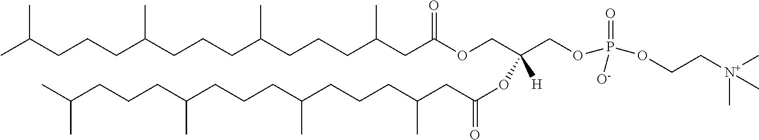 [SMILES: CC(C)CCCC(C)CCCC(C)CCCC(C)CC(=O)OC[C@H](COP(=O)([O-])OCC[N+](C)(C)C)OC(=O)CC(C)CCCC(C)CCCC(C)CCCC(C)C]

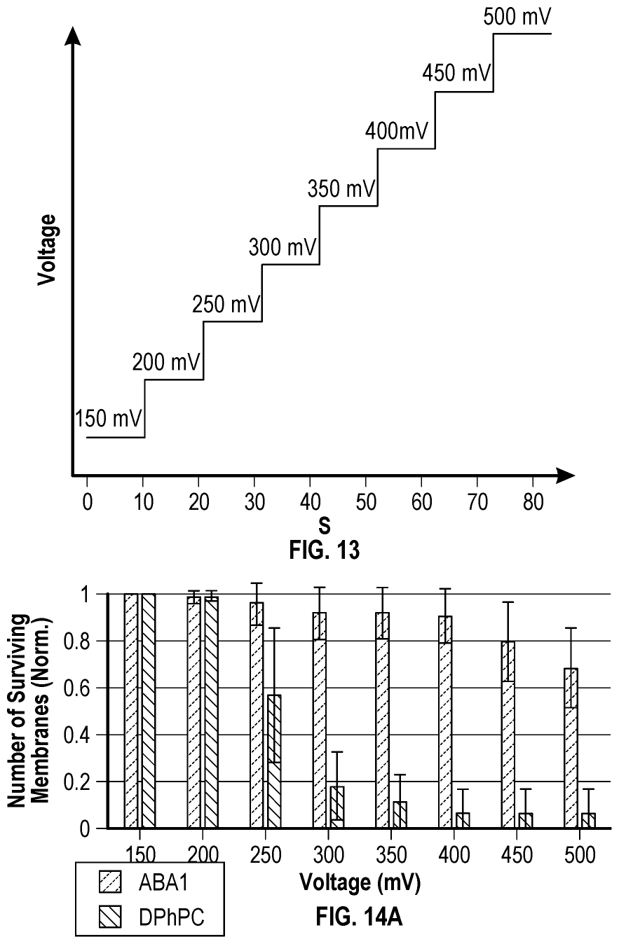

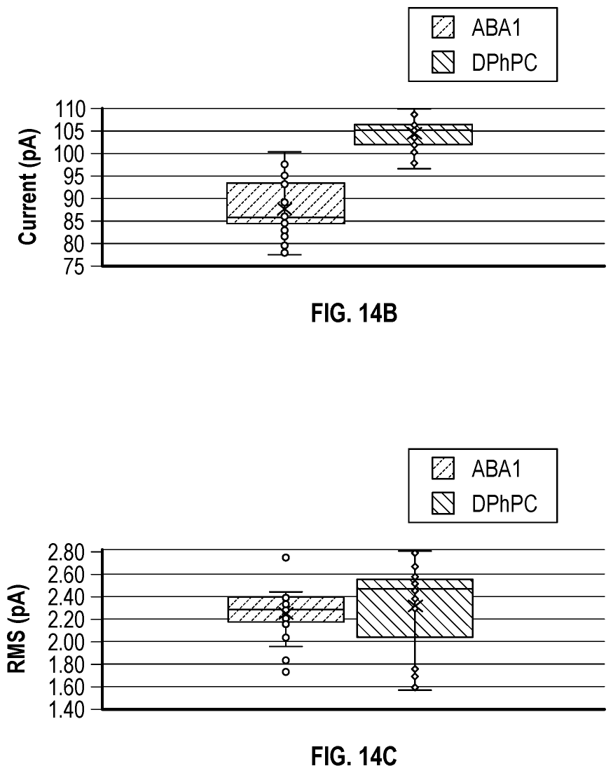

FIG. 14A illustrates a plot of the measured breakdown voltage of example barriers, namely barriers formed using ABA1 and DPhPC. This test is used as a stability metric, with a higher breakdown voltage being associated with greater mechanical stability of membranes, and thus being better for use where membranes are exposed to extreme conditions (such as sequencing) for long periods of time. As may be understood from FIG. 14A, the barriers formed using ABA1 had significantly higher stability than those using DPhPC as voltage increased. FIG. 14B illustrates a plot of the respective currents through the barriers of FIG. 14A with an MspA nanopore inserted therein. FIG. 14C illustrates a plot of the respective noise in current through the barriers of FIG. 14A with the MspA nanopore inserted therein. From FIGS. 14A-14C it may be understood that although DPhPC offers relatively high pore current values and low noise values, it exhibits relatively low breakdown voltage, limiting its widespread use and reducing the longevity useful for sequencing. In comparison, ABA1 shows lower pore current values and higher noise values, but a significantly higher breakdown voltage, with about 80% of membranes remaining at 450 mV.

Additional barriers were formed using the following ABA polymers with COOH end groups (ABA2 and ABA3, respectively):

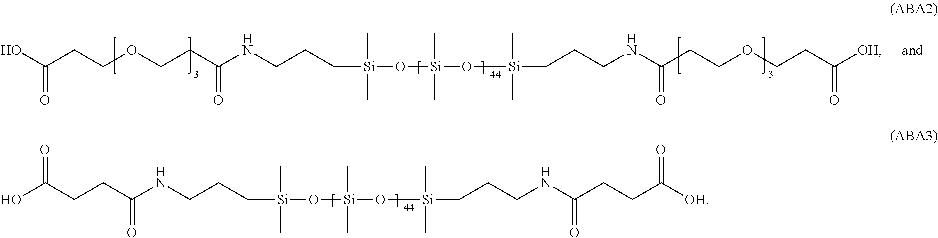

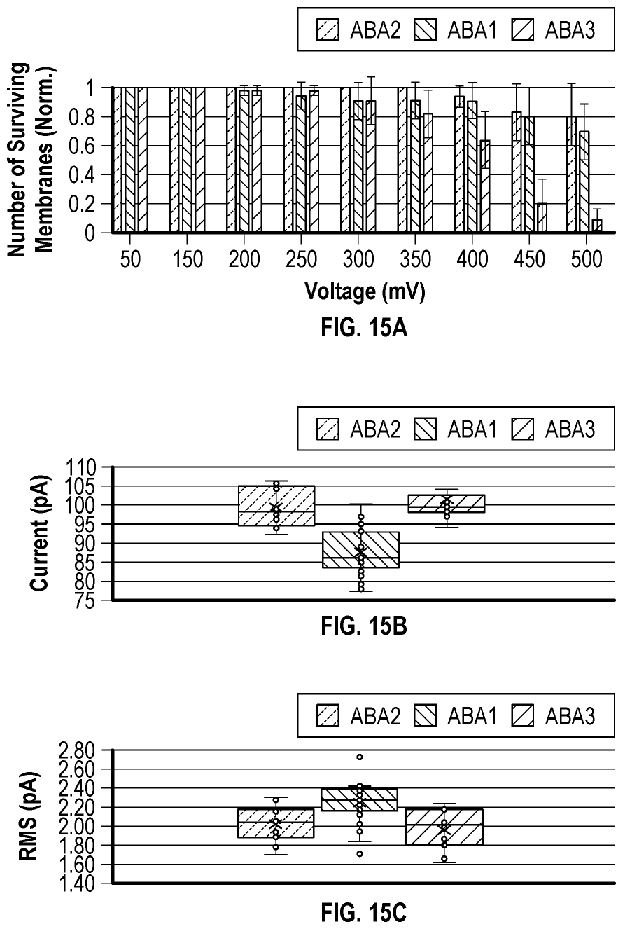

The performance of barriers formed using ABA2 and ABA3 polymers was assessed and compared to that of ABA1 as shown in FIGS. 15A-15C. More specifically, FIG. 15A illustrates a plot of the measured breakdown voltage of additional example barriers, namely barriers formed using ABA1, ABA2, and ABA3. FIG. 15B illustrates a plot of the respective currents through the barriers of FIG. 15A with an MspA nanopore inserted therein. FIG. 15C illustrates a plot of the respective noise in current through the barriers of FIG. 15A with an MspA nanopore inserted therein. Both ABA2 and ABA3 were both able to form stable membranes. ABA3 showed breakdown voltage that was increased over DPhPC, but lower than ABA1. However, the MspA pore current values seen for ABA3 membranes were high and noise values were low, making it comparable to DPhPC in these metrics. ABA2 showed outstanding breakdown voltage with about 84% of membranes remaining at 450 mV, with high pore current values and low noise values, comparable to DPhPC.

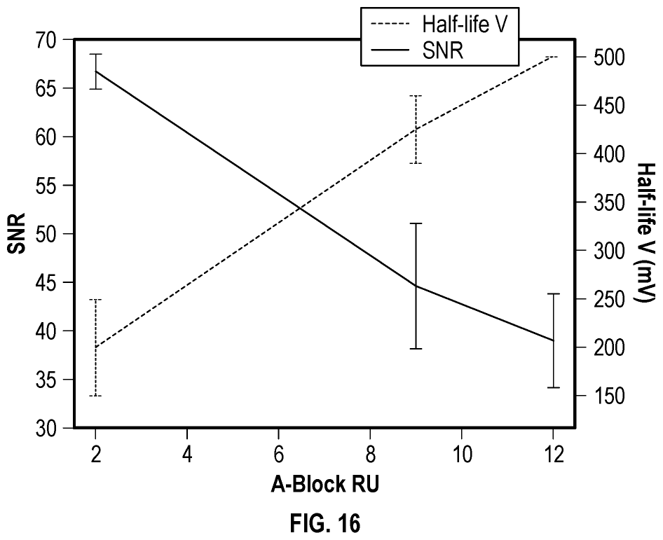

From FIGS. 14A-14C and 15A-15C, it may be understood that reducing the size of the hydrophilic block may significantly increase nanopore current values and reduce nanopore noise values. The polymers used as the hydrophilic and/or hydrophobic blocks, respective lengths thereof, and end groups may be co-selected so as to provide barriers that are of similar stability as barriers with larger hydrophilic blocks while having suitable fluidity to allow facile nanopore insertion. FIG. 16 illustrates a plot of barrier noise and half-life voltage as a function of the number of repeat units (RUs) in the hydrophobic A block. More specifically, FIG. 16 illustrates measurements from a titration of ABA polymers having the same hydrophobic block (PDMS) size, but varying hydrophilic block (PEO) units, with respect to the stability to breakdown voltage test and the signal to noise (current/noise) ratio. The blue line shows increasing stability to breakdown voltage reported as half-life of membranes (voltage at which more than 50% of membranes break); the red line shows the decrease of the signal to noise (current/noise) ratio with the number of hydrophilic repeat units. It is believed that similar the results will translate across to other hydrophobic-hydrophilic block chemistries.

### Example 2

The performance of different copolymers was assessed in terms of ease of membrane preparation, controlled single vs. multiple nanopore insertion, and stability against osmotic pressure.

All materials were tested on the Orbit-16 instrument from NanION. This tool allows mechanical painting by rotation of a Teflon stirring bar on top of the chip cavities as well as electrical testing of the membrane/pore construct (membrane capacitance measurement, nanopore I/V curve).

Membrane Painting and Nanopore Insertion

Copolymers listed in Table 1 below respectively were dissolved in an octane:butanol (95:5 vol) solvent mixture at a concentration of 5 mg/mL prior to testing through suspended membrane formation (also called membrane painting) using a support including a circular aperture such as described with reference to 2A-2B, 3-6, and 18.

TABLE 1

| | End | | |
|---|---|---|---|
| Polymer | group(s) | Label | Structure |
| PEO-b- PIB-b- PEO | —CH₃ | ABA4 | 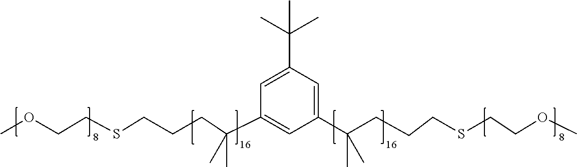 |
| PEO-b- PIB-b- PEO | —COOH | ABA5 | 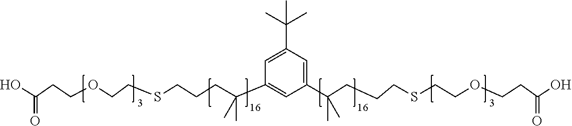 |
| PEO-b- PIB-b- PEO | —COOH | ABA6 | 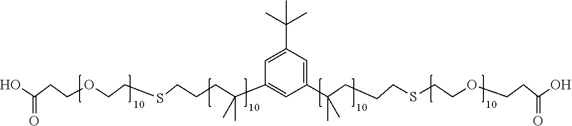 |
| PEO-b- PDMS-b- PEO | —COOH | ABA7 | 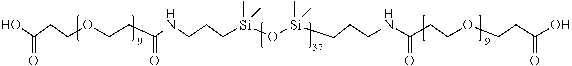 |
| PEO-b- PIB | —CH₃ | AB1 | 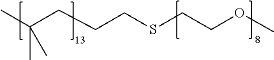 |
| PEO-b- PIB | —CH₃ (on PIB) and —COOH (on PEO) | AB2 | 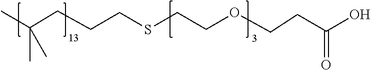 |
| PEO-b- PIB | —CH₃ (on both PIB) and PEO) | AB3 | 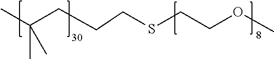 |
| PEO-b- PIB | —CH₃ (on PIB) and —COOH (on PEO) | AB4 | 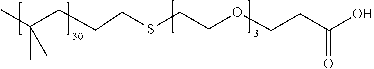 |
| PEO-b- PBD | —CH₃ (on PBD) and —OH (on PEO) | P5 | 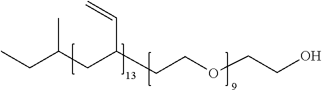 |

Characterization tests were used to extract metrics that are believed to be relevant to the nanopore-sensing application of such membranes. Those metrics fall under categories such as stability (e.g., resilience of membranes/membranes-pore system against stress tests, including accelerated tests, sequencing conditions), throughput (e.g., membrane painting yield, pore insertion and retention yields) and quality (e.g., membrane-pore current and noise level and consistency, SNR, electrical insulation/leakiness of membrane).

The first characterization tests that were carried out focused on success rate in membrane formation (membrane painting yields), membrane resistance to breakdown voltage, biological pore insertion (MspA pores), resulting current and noise of said pore inside of the block copolymer membranes. These helped to assess the performance of the PIB-PEO-based membranes to one another and to membranes formed with other polymers.

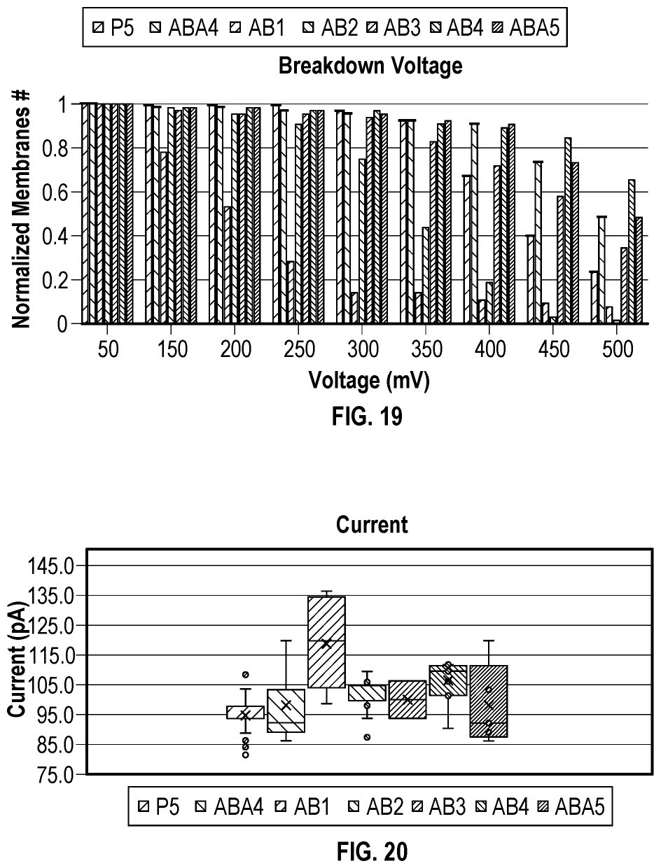

In terms of painting quality, all the PIB-b-PEO block copolymers (AB1, AB2, AB3, AB4) and PEO-b-PIB-b-PEO block copolymers (ABA4, ABA5, ABA6) could be painted to form a suspended membrane. Particularly satisfactory performance was achieved by the PEO-b-PIB-b-PEO block copolymers with an ABA architecture. For example, FIG. 19 illustrates a plot describing the breakdown voltage measured for membranes formed using P5, ABA4, AB1, AB2, AB3, AB4, and ABA5. For the membrane formed using P5, it may be seen in FIG. 19 that at voltages of about 300 mV and below, the normalized number of membranes which remained substantially intact ranged from about 1.0 at 0 V to about 0.95 at 300 mV; and that at voltages of about 350 mV and above, the normalized number of membranes decreased from about 0.9 at 350 mV to about 0.22 at 500 mV. For the membrane formed using ABA4, it may be seen in FIG. 19 that at voltages of about 300 mV and below, the normalized number of membranes which remained substantially intact ranged from about 1.0 at 0 V to about 0.95 at 300 mV; and that at voltages of about 350 mV and above, the normalized number of membranes decreased from about 0.9 at 350 mV to about 0.5 at 500 mV. For the membrane formed using ABA5, it may be seen in FIG. 19 that at voltages of about 300 mV and below, the normalized number of membranes which remained substantially intact ranged from about 1.0 at 0 V to about 0.95 at 300 mV; and that at voltages of about 350 mV and above, the normalized number of membranes decreased from about 0.9 at 350 mV to about 0.5 at 500 mV.

For the membrane formed using AB1, it may be seen in FIG. 19 that at voltages of about 300 mV and below, the normalized number of membranes which remained substantially intact ranged from about 1.0 at 0 V to about 0.16 at 300 mV; and that at voltages of about 350 mV and above, the normalized number of membranes decreased from about 0.16 at 350 mV to about 0.04 at 500 mV. For the membrane formed using AB2, it may be seen in FIG. 19 that at voltages of about 300 mV and below, the normalized number of membranes which remained substantially intact ranged from about 1.0 at 0 V to about 0.75 at 300 mV; and that at voltages of about 350 mV and above, the normalized number of membranes decreased from about 0.45 at 350 mV to about 0.01 at 500 mV. For the membrane formed using AB3, it may be seen in FIG. 19 that at voltages of about 300 mV and below, the normalized number of membranes which remained substantially intact ranged from about 1.0 at 0 V to about 0.9 at 300 mV; and that at voltages of about 350 mV and above, the normalized number of membranes decreased from about 0.85 at 350 mV to about 0.035 at 500 mV. For the membrane formed using AB4, it may be seen in FIG. 19 that at voltages of about 300 mV and below, the normalized number of membranes which remained substantially intact ranged from about 1.0 at 0 V to about 0.97 at 300 mV; and that at voltages of about 350 mV and above, the normalized number of membranes decreased from about 0.9 at 350 mV to about 0.65 at 500 mV.

As may be understood from the plot shown in FIG. 19, the ABA block copolymers ABA4 and ABA5, and the AB4 block copolymer, showed particularly high resilience to the breakdown voltage stress test.

Another difference between membranes is how difficult it was to insert a single nanopore into that membrane. For nanopore sequencing such as described with reference to FIGS. 9-12 and 17, it is useful to insert a single nanopore in each membrane.

FIG. 20 illustrates a plot of MspA nanopore/membrane construct stability in 1M KCl+50 mM HEPES buffer. For the membrane formed using P5 with an MspA nanopore inserted therein, it may be seen in FIG. 20 that at a voltage of 100 mV, the membrane-pore construct had a current which ranged between about 92 pA and about 97 pA. For the membrane formed using ABA4 with an MspA nanopore inserted therein, it may be seen in FIG. 20 that at a voltage of 100 mV, the membrane-pore construct had a current which ranged between about 89 pA and about 104 pA. For the membrane formed using ABA5 with an MspA nanopore inserted therein, it may be seen in FIG. 20 that at a voltage of 100 mV, the membrane-pore construct had a current which ranged between about 87 pA and about 110 pA. For the membrane formed using AB1 with an MspA nanopore inserted therein, it may be seen in FIG. 20 that at a voltage of 100 mV, the membrane-pore construct had a current which ranged between about 104 pA and about 135 pA. For the membrane formed using AB2 with an MspA nanopore inserted therein, it may be seen in FIG. 20 that at a voltage of 100 mV, the membrane-pore construct had a current which ranged between about 100 pA and about 105 pA. For the membrane formed using AB3 with an MspA nanopore inserted therein, it may be seen in FIG. 20 that at a voltage of 100 mV, the membrane-pore construct had a current which ranged between about 94 pA and about 107 pA. For the membrane formed using AB4 with an MspA nanopore inserted therein, it may be seen in FIG. 20 that at a voltage of 100 mV, the membrane-pore construct had a current which ranged between about 96 pA and about 110 pA.

The ABA5 and ABA6 membranes were identified as having particularly good performance. A notable improvement shown in ABA5 is the enhancement of membrane resilience. A notable improvement shown in ABA6 is the enhancement of the insertion and retention of single MspA nanopores into the membrane, with a lower variability. Various properties of the membranes are shown in the table below.

TABLE 2

| Category | Property | ABA7 | ABA5 | ABA6 |
|---|---|---|---|---|
| Membrane | Membrane survival | 95% | 100% | 100% |
| quality | rate at 450 mV (%) | | | |
| MspA | Open Pore Current | 104 pA | 103 pA | 104 pA |
| nanopore | (50 um wells, | | | |
| current | 100 mV) (pA) | | | |
| | Membrane-Pore (MP) | 2.13 pA | 1.46 pA | 1.62 pA |
| | RMS noise (pA) | | | |
| | Pore + membrane | 49 | 71 | 64 |
| | SNR | | | |
| Yields | Membrane | >95% | >95% | >95% |
| | painting yield | | | |
| | (number of membranes | | | |
| | formed, divided | | | |
| | by number | | | |
| | of attempts to form | | | |
| | membranes) (%) | | | |
| | Pore insertion voltage | 500-850 | 800-1000 | 350-450 |
| | (mV) | mV | mV | mV |
| | Single Pore (SP) | >95% | ~90% | >95% |
| | percentage (after | | | |
| | insertion step) (%) | | | |
| | SP Survival Rate under | >95% | >95% | >95% |
| | flush (3 × 250 uL) (%) | | | |
| MspA | Open Pore current | 36.18 pA | 35.02 pA | 36.04 pA |
| nanopore | (400 mM KCl, 50 mV, | | | |
| current + | 50 um wells) (pA) | | | |
| con- | SP current std dev | 1.93 pA | 0.8 pA | 0.4 pA |
| sistency | (within 1 exp) (±pA) | | | |
| Resilience | 700 to 1200 mV/10 us- | ~65% | ~ 95% | ~95% |
| against | membrane survival at | | | |
| bio- | 1200 mV (%) | | | |
| chemistry | 700 to 1200 mV/10 us- | >1200 mV | >1200 mV | >1200 mV |
| | voltage at | | | |
| | 50% membrane | | | |
| | survival (mV) | | | |
| | 700 to 1200 mV/10 us- | 1000 mV | 1000 mV | 900 mV |
| | voltage at | | | |
| | 50% membrane | | | |
| | and single pore survival | | | |
| | (mV) | | | |

Membrane quality was measured by membrane survival rate at 450 mV of current across the barrier. Both ABA5 and ABA6 had a 100% survival rate, and ABA7 had an approximately 95% survival rate.

The current through the membrane when an MspA nanopore was inserted was also measured. The open pore current was measured at a voltage across the barrier of 100 mV. ABA5 and ABA6 had similar currents of 103 pA and 104 pA, respectively, and ABA7 had a current of 104 pA. The root mean square (RMS) average of the current noise through the barrier was also measured in a similar fashion, after inserting the MspA nanopore into the membrane. ABA5 and ABA6 had similar RMS current noise averages of 1.46 pA and 1.62 pA, respectively, and ABA7 had a RNS current noise of 2.13 pA. The signal-to-noise ratio (SNR) of current through the barrier was also measured in a similar fashion. ABA5 and ABA6 had SNRs of 71 and 64, respectively, and ABA7 had a SNR of 49.

Various yield percentages were also measured. ABA5, ABA6, and ABA7 all had a membrane painting yield of greater than 95%. The voltage required to insert an MspA nanopore into the membrane was also measured in a similar fashion. ABA5 required a voltage between about 800 mV and about 1000 mV, while ABA6 required a voltage between about 350 mV and 450 mV, and ABA7 required a voltage between about 500 mV and about 850 mV. After a nanopore insertion step was carried out, the percentage of membranes containing an MspA nanopore was about 90% for ABA5, greater than 95% for ABA6, and greater than 95% of ABA7. The percentage of single-pore membranes surviving a flush was also measured in a similar fashion. Specifically, the membranes were flushed with 250 μL of fluid three times before the survival rate was determined. Both ABA5 and ABA6 had survival rates of greater than 95%, as did ABA7.

The tightness of the open pore current spread of MspA nanopores within the membrane were also measured. Specifically, the current was measured 400 mM KCl and applying a current of 50 mV across the membrane. ABA5 and ABA6 had open pore currents of 35.02 pA and 36.04 pA, respectively, and ABA7 had an open pore current of 36.18 pA. The standard deviation of the current was measured in a similar fashion. ABA5 and ABA6 had standard deviations of 0.8 pA and 0.4 pA, respectively, and ABA7 had a standard deviation of 1.93 pA.

The resilience of the barriers was also measured. The membrane survival rate was measured after the barrier was subjected to a waveform which was made of a train of positive voltage micro pulses, spaced by negative voltage periods at −100 mV for 100 ms. The train of positive voltage pulses had a total of 20 pulses, with duration of 10 μs. The spacings between them had a set duration value of 30 ms and a voltage held at +50 mV. During a first cycle, the waveform was applied continuously for a period of 5 minutes and the magnitude of the pulses kept at +700 mV. In further applied cycles (applied again for 5 mins each), the pulsing intensity was increased from +700 mV to +1200 mV in 100 mV steps, for a total of six cycles. Both ABA5 and ABA6 had a survival rate, after application of the described waveform, of about 95%, while ABA7 had a survival rate of about 65%. In the same experiment/test, the voltage at which 50% of the membranes survived after being subjected to said waveforms was determined. For ABA5, ABA6, and AB4, the voltage was determined to be greater than 1200 mV. In another test/experiment, the same waveform cycle/test was repeated, but using membranes after having a single MspA pore inserted. For ABA5 and ABA7, the voltage was determined to be about 1000 mV, while for ABA6, the voltage was determined to be about 900 mV.

Based on the different metrics selected to assess fitness for use in polymeric membranes for nanopore sequencing applications, these results demonstrate that the performance of membranes including PIB as the hydrophobic B block are particularly suitable for use in such applications. For example, the different metrics indicate that ABA and AB copolymers using PIB as the hydrophobic B block, particularly those including —COOH as the end group, can form membranes with useful properties for nanopore sequencing applications, including relatively high membrane formation yield, relatively high pore insertion and retention yield, relatively high stability under sequencing conditions, and relatively good electrical properties for high read quality. The inventors believe that such properties are likely to be correlated with ease of flow-cell fabrication, instrument throughout, instrument/chip reliability, and high read accuracy, which may be important to commercial manufacture and use of nanopore sequencing devices, e.g., such as described with reference to FIGS. 9-12 and 17.

Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A barrier between first and second fluids, the barrier being suspended by a barrier support defining an aperture, the barrier comprising:
one or more layers suspended across the aperture and comprising molecules of a block copolymer,
each molecule of the block copolymer comprising one or more hydrophilic blocks having an approximate length A and one or more hydrophilic blocks having an approximate length B,
the hydrophilic blocks forming outer surfaces of the barrier and the hydrophobic blocks being located within the barrier; and end groups coupled to ends of the hydrophilic blocks that form outer surfaces of the barrier, the end groups having a different hydrophilicity than the hydrophilic blocks.

2. The barrier of claim 1, wherein the end groups are selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

3. The barrier of claim 1, wherein the hydrophobic blocks comprise a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

4. The barrier of claim 1, wherein the block copolymer is a diblock copolymer.

5. The barrier of claim 4, wherein the hydrophobic block is polybutadiene (PBd).

6. (canceled)

7. The barrier of claim 1, wherein the block copolymer is a triblock copolymer having two hydrophilic blocks and one hydrophobic block.

8. The barrier of claim 5, wherein the hydrophobic block is poly(isobutylene) (PIB).

9. (canceled)

10. (not entered)

11. The barrier of claim 1, wherein the block copolymer is a triblock copolymer having two hydrophobic blocks and one hydrophilic block.

12. (canceled)

13. The barrier of claim 1, further comprising a nanopore disposed therein and providing contact between the first fluid and the second fluid.

14. A barrier comprising:
at least one layer comprising a plurality of molecules,
each of the molecules comprising first and second hydrophilic blocks, first and second end groups, and a hydrophobic block,
the hydrophobic block being disposed between and coupled to the first and second hydrophilic blocks, and
the first and second end groups respectively being coupled to ends of the first and second hydrophilic blocks and having a different hydrophilicity than the first and second hydrophilic blocks,
the first and second end groups forming outer surfaces of the barrier, and
the hydrophobic blocks being within the barrier.

15. The barrier of claim 14, wherein the first and second hydrophilic blocks each comprise poly(ethylene oxide) (PEO).

16. The barrier of claim 15, wherein the first and second hydrophilic blocks each comprise between about 2 and about 12 PEO repeating units.

17. The barrier of any one of claims 14 to 16, wherein the hydrophobic block comprises poly(dimethylsiloxane) (PDMS) or poly(isobutylene) (PIB).

18. The barrier of claim 17, wherein the hydrophobic block comprises about 13 to about 44 PDMS repeating units.

19. The barrier of claim 17, wherein the hydrophobic block comprises about 13 to about 44 PIB repeating units.

20. The barrier of claim 14, wherein the first and second end groups independently are selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

21. The barrier of claim 14, further comprising a nanopore.

22. (canceled)

23. A barrier comprising:
at least one layer comprising a plurality of molecules,
each of the molecules comprising first and second ionic end groups and a hydrophobic block, the hydrophobic block being disposed between and coupled to the first and second ionic end groups;
the ionic end groups forming first and second outer surfaces of the barrier, and
the hydrophobic blocks being within the barrier.

24-35. (canceled)

36. A barrier comprising:
a first layer comprising a first plurality of molecules,
each of the molecules comprising a hydrophilic block, a hydrophobic block, and an end group,
the hydrophilic block being coupled to the hydrophobic block, and
the end group being coupled to an end of the hydrophilic block and having a different hydrophilicity than the hydrophilic block; and
a second layer comprising a second plurality of the molecules,
the end groups forming first and second outer surfaces of the barrier, and the hydrophobic blocks of the first and second pluralities of the molecules contacting one another within the barrier.

37-44. (canceled)

45. A barrier comprising:
a first layer comprising a first plurality of molecules,
each of the molecules comprising first and second hydrophobic blocks, a hydrophilic block, and first and second end groups,
the hydrophilic block being disposed between and coupled to the first and second hydrophobic blocks,
the first and second end groups respectively being coupled to ends of the first and second hydrophobic blocks and having a different hydrophobicity than the first and second hydrophobic blocks; and
a second layer comprising a second plurality of the molecules,
the hydrophilic blocks of the first plurality of the molecules forming a first outer surface of the barrier,
the hydrophilic blocks of the second plurality of the molecules forming a second outer surface of the barrier, and
the first and second end groups of the first and second pluralities of the molecules contacting one another within the barrier.

46-54. (canceled)
